(19) United States
(12) Patent Application Publication
(10) Pub. No.: US 2023/0044025 A1
(43) Pub. Date: Feb. 9, 2023

(54) SYSTEM AND METHOD OF TREATING OR PREVENTING RESPIRATORY FAILURE WITH AEROSOLIZED COLLAGENASE

(71) Applicant: Charles Nathan Trujillo, Morrisville, NC (US)

(72) Inventor: Charles Nathan Trujillo, Morrisville, NC (US)

(21) Appl. No.: 17/879,050

(22) Filed: Aug. 2, 2022

Related U.S. Application Data
(60) Provisional application No. 63/230,411, filed on Aug. 6, 2021.

(51) Int. Cl. A61K 38/57; A61K 9/00; A61P 11/00

(52) U.S. Cl. CPC A61K 38/57; A61K 9/0078; A61P 11/00

(57) ABSTRACT

Methods for treating and/or alleviating acute respiratory distress syndrome in a patient diagnosed with or at risk of developing acute respiratory distress syndrome are disclosed. The methods comprise administering a therapeutically effective amount of an aerosolized collagenase to the patient, wherein the collagenase breaks down scar tissue in the lung, thereby treating the ARDS, or delaying or preventing onset of ARDS.

### CROSS-REFERENCE TO RELATED APPLICATIONS

This application claims priority to U.S. Provisional Patent Application No. 63/230,411, filed Aug. 6, 2021, the entire content of which is hereby incorporated by reference.

### TECHNICAL FIELD

The presently disclosed subject matter is generally directed to a system and method of treating or preventing respiratory failure with aerosolized collagenase. More particularly, the present invention refers to use of CCol in a novel nebulized form for the treatment of pulmonary fibrotic diseases and ARDS.

### BACKGROUND

Acute respiratory distress syndrome (ARDS) is a critical illness characterized by acute lung injury leading to pulmonary edema and respiratory failure. Patients that have experienced trauma, hemorrhage, aspiration of gastric content, severe pneumonia, influenza, and/or sepsis are at risk of developing ARDS. In addition, ARDS is also the proximal cause of morbidity in a large percentage of patients with upper respiratory tract infections, such as Severe Acute Respiratory Syndrome (SARS) caused by coronavirus SARS-CoV and Middle East Respiratory Syndrome (MERS) thought to be caused by coronavirus MERS-CoV. Current therapies focus on treating the cause of the injury or disease, such as through the administration of antibiotics for infection, addressing fluid accumulation, and reversing the associated reductions in oxygen transport in the alveoli. Despite significant advances in critical care management, mortality from ARDS remains over 35%. Accordingly, it would be beneficial to provide an improved system and method for treating ARDS, for treating the associated inflammatory process, and for improving the tissue repair process in the lungs.

### SUMMARY

The present invention is a novel treatment for ARDS/fibrotic lung pathology via the use of Ccol in a nebulized form. This invention provides a solution to the virtual lack of therapeutic options to treat ARDS/fibrotic lung diseases. This invention is aimed at reducing the excessive collagen deposition associated with ARDS secondary to local deficiency in native MMP at the alveolar lung surface interphase level. We hypothesize that nebulized Ccol will reduce excessive ECM deposition, improve pulmonary mechanics, enhance oxygenation and subsequent fibrosis, to accomplish this the following will be performed.

In some embodiments, the presently disclosed subject matter is directed to a method of treating acute lung injury in a patient. Particularly, the method comprises administering by inhalation a therapeutically effective amount of one or more aerosolized collagenases to the lung tissue of the patient in need thereof such that the lung injury is treated. The one or more collagenases are administered at a dose of about 10-300 U/kg of body weight. The one or more collagenases are administered to the lungs at the site of the proximate acute injury.

In some embodiments, the presently disclosed subject matter is directed to a method of preventing acute lung injury in a patient. Particularly, the method comprises administering by inhalation a therapeutically effective amount of one or more aerosolized collagenases to the lung tissue of the patient such that the lung injury is prevented. The one or more collagenases are administered at a dose of about 10-300 U/kg of body weight. The one or more collagenases are administered to the lungs at the site of the proximate acute injury.

In some embodiments, the acute lung injury is acute respiratory distress syndrome (ARDS).

In some embodiments, the acute lung injury is selected from COPD, lung cancer, asthma, cystic fibrosis, emphysema, bronchitis, bronchiectasis, interstitial lung disease, interstitial fibrosis, bacterial pneumonia, viral pneumonia, fungal pneumonia, parasitic pneumonia, mycobacteria-caused pneumonia, occupational lung diseases, systemic lupus erythematosus, rheumatoid arthritis, scleroderma, dermatomyositis, mixed connective tissue disorder, vasculitis associated lung disease, sarcoid, or combinations thereof.

In some embodiments, the acute lung injury is the result of sepsis, pancreatitis, trauma to the lung tissue, pneumonia, aspiration, COVID-related illness, or combinations thereof.

In some embodiments, the administering is by nasal or oral inhalation.

In some embodiments, the collagenase acts as an enzymatic debrider, removing dead tissue from the lungs.

In some embodiments, the aerosolized collagenase has a diameter of about 0.1-10 μm.

In some embodiments, the dosage of aerosolized collagenase administered is about 0.1, 0.5, 1, 2, 3, 4, 5, 6, 7, 8, 9, or 10 mg/kg of patient body weight.

In some embodiments, the one or more collagenase is administered using an ultrasonic nebulizer.

In some embodiments, the patient is a human.

In some embodiments, the patient is a human susceptible to developing acute lung injury.

In some embodiments, the collagenase is selected from one or more of MMP1, MMP2, MMP3, MMP7, MMP8, MMP9, MMP10, MMP11, MMP12, MMP13, MMP14, MMP15, MMP16, MMP17, and microbial MMPs.

In some embodiments, the presently disclosed subject matter is directed to a kit comprising a therapeutically effective amount of an aerosol form of collagenase and instructions for use.

In some embodiments, the kit further includes a nebulizer system selected from a jet aerosol, an ultrasonic nebulizer, or a dry powder inhalation system.

### BRIEF DESCRIPTION OF THE DRAWINGS

FIG. 1 is a schematic of a method of treating a patient afflicted with a lung disorder in accordance with some embodiments of the presently disclosed subject matter.

FIG. 2 is a method of preventing affliction of a lung disorder in a susceptible patient in accordance with some embodiments of the presently disclosed subject matter.

FIG. 3 is an electron microscope image of normal alveolar structure.

FIGS. 4a and 4b are representations depicting the organizational structure of the alveolar and lung lining fluid.

FIG. 5 is a table illustrating characteristics of human lung lining fluid in the conducting airways and the respiratory zone.

FIG. 6 is an illustration of the organization of these various transporters involved in maintaining the AVSF.

FIG. 7 is a depiction of normal wound healing requiring sequential ECM degradation and resorption.

FIG. 8 is a table of the various types of collagens, their function, and relative distribution throughout the body.

FIG. 9 is a depiction of collagen assembly to form collagen fiber.

FIG. 10 is a table of the various classes of MMP's and their respective function.

FIGS. 11a-11d are images of abnormal collagen deposition observed in normal skin, a normotrophic scar, a hypertrophic scar, and a keloidal scar, respectively.

FIG. 12 is a table illustrating the relationship between clinical classification and Type III Collagen Proportion.

FIG. 13a is a table summarizing histological changes in ARDS.

FIG. 13b is a graph illustrating the course of histologic events in DAD depicted as days following lung injury versus percentage of maximum.

FIG. 14 is a photomicrograph of acute phase DAD (original magnification ×200 H-E stain), showing characteristic hyaline membranes at the arrows and alveolar wall edema in acute phase DAD. Capillary leak has resulted in amorphous eosinophilic edema fluid in the alveolar spaces.

FIGS. 15a and 15b are photomicrographs (original magnifications ×320 (FIG. 15a) and ×100 (FIG. 15b) in H-E stain in the same patient showing organizing fibroblastic tissue as plugs within the alveolar spaces (arrows in FIG. 15a and diffusely involving the interstitium (stars in FIG. 15b).

FIG. 16a is a low magnification image showing extensive interstitial fibroblastic proliferation (granulation tissue) producing marked thickening of the alveolar septa.

FIG. 16b is a high power image of thickened alveolar septa due to a fibroblastic proliferation associated with hyperplastic alveolar pneumocytes.

FIGS. 17a and 17b are high magnification images with cells showing a high nucleocytoplasmic ratio, hyperchromasia, and irregular nuclear membrane.

FIG. 18a is a photomicrograph (medium power) of hyaline membranes incorporated into the alveolar septa.

FIG. 18b is a high power photomicrograph showing epithelium growing over hyaline membrane that is being incorporated into the alveolar septa.

FIGS. 19a-19c are graphs of total collagen versus duration of lung disease.

FIGS. 20a and 20b are illustrations of lung function during inspiration and after expiration.

FIGS. 21a-21c are representations of the interdependence of alveolar units, negative pressure breathing, and positive pressure ventilation.

FIG. 22a is a unified processing model of triple helical and microfibrillar collagen. A collagen triple helix initially docks to the peptidase domain of collagenase.

FIG. 22b is a unified processing model of triple helical and microfibrillar collagen showing step 2, closed conformation, showing the activator HEAT repeats interacting with the triple helix, a prerequisite for collagen hydrolysis.

FIG. 22c is a unified processing model of triple helical and microfibrillar collagen showing step 3, semi-open conformation, allowing for exchange and processive degradation of all three alpha chains.

FIG. 22d is a unified processing model of triple helical and microfibrillar collagen showing collagenase with a docked collagen microfibril.

FIG. 22e is a unified processing model of triple helical and microfibrillar collagen showing step 2, closed conformation with all triple helices but one being expelled from the collagenase.

FIG. 22f is a unified processing model of triple helical and microfibrillar collagen showing step 3, semi-open conformation allowing for complete processing of the triple helix. The collagenase will then relax back to the open state and only then allow the remaining part of the microfibril to enter the collagenase.

FIG. 23 is a representation of cleavage sites in collagen I by MMP-1(delta C), MMP-3(delta C) and HLE detected in the presence of MMP-1 (E200A).

FIGS. 24a-24c are schematics showing the sites of hydrolysis (vertical arrows) of type I, II, and III collagens by the class I CHC, and a degradation scheme for each.

FIGS. 25a-25c are schematics showing the sites of hydrolysis (vertical arrows) of type I, II, and III collagens by the class II CHC, and a degradation scheme for each.

FIG. 26a illustrates a jet nebulizer that delivers compressed gas through a jet, causing a region of negative pressure. The solution or suspension to be aerosolized in entrained into the gas stream and is sheared into a liquid film. The film is unstable and breaks into droplets due to surface tension forces. A baffle in the aerosol stream produces smaller particles.

FIG. 26b illustrates an ultrasound nebulizer where an alternating electric field is applied to a piezoelectric transducer that converts the electrical signal into a periodic mechanical vibration. The vibrations are transmitted through a buffer to the drug solution a form a fountain of liquid in the nebulization chamber. A baffle is used to reduce droplet size of the aerosol.

FIG. 26c illustrates a vibrating mesh nebulizer where contraction and expansion of a vibrational element produce an upward and downward movement of the aperture plate. The holes of the mesh have a tapered shape with a larger cross-section on the liquid side and a smaller cross-section on the side the droplets emerge. Aerosol particle size and flow are determined by the exit diameter of the aperture holes.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures. It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

Clostridium Histolyticum derived Collagenase (CCol), has been utilized to treat imbalances of collagen deposition for various medical conditions [1] [2]. Its use has demonstrated the propensity to accelerate human dermal wound healing [2] [3] [4] [5], hasten inflammation [6], with extensive evidence supporting its safety [2] [5] [4]. In relation, ARDS (Acute Respiratory Distress Syndrome) is characterized by inflammatory destruction of alveoli, propagating fibrosis of lung parenchyma [7] [8], of which is largely related to an imbalance in collagen turnover [9] [8]. This disease continues to have poor long-term survival [10] and quality of life (QOL) [11], with mortality rates as high as 40% [12]. Additionally, the inflammatory response leads to excessive lung collagen deposition of which is strongly associated with the poor outcomes evident in ARDS [13] [14] [15]. Numerous in-vivo studies in humans and in mice, however, have shed light on this matter, attributing the imbalance to deficiencies in alveolar collagenases/matrix metalloproteinases (MMP), with virtual absence of MMP's at the extracellular matrix (ECM) interphase on immunohistochemical analysis [16] [17] [9] [8] [18] [19]. CCol's substrate specificity has considerable overlap with mammalian MMP's, but unlike mammalian MMPs, CCol is relatively incapable of degrading viable tissue[20] [2, 21] [5] [4] [22] [23]. Furthermore, in-vivo and in-vitro evidence demonstrates that CCol can effectively bind all types of human collagen, irrespective of organ site, including the lung [24]. Thus, given the evidence provided, supplemental CCol in a novel nebulized form is believed to alter the alveolar remodeling micro-environment, limit excessive collagen deposition, and subsequent fibrosis in ARDS. It is expected that the use of the novel application of CC will improve ARDS mortality, long-term survival and QOL.

The instant disclosure is therefore based on the discovery that aerosolized collagenase is effective in treating lung disorders. Specifically, the presently disclosed subject matter is generally directed to a method for treating acute respiratory distress syndrome (ARDS) in a patient and/or treating a patient at risk for developing acute respiratory distress syndrome (ARDS). ARDS is typically provoked by an acute injury to the lungs, such as sepsis, pancreatitis, trauma, pneumonia, aspiration, as well as COVID-related illnesses. The underlying mechanism of ARDS involves diffuse injury to the cells that form the barrier of the microscopic air sacs of the lungs, surfactant dysfunction, activation of the immune system, and dysfunction of the body's regulation of blood clotting. ARDS can be characterized by the influx of protein rich edema fluid into the air spaces due to increased permeability of the alveolar capillary barrier. Fluid build-up in the lungs leads to impaired gas exchange and occurs with concurrent systemic release of inflammatory mediators, causing inflammation, hypoxemia, and frequently multiple organ failure. In effect, ARDS impairs the ability of the lungs to exchange oxygen and carbon dioxide.

ARDS afflicted over 550,000 patients in the United States in 2020, leading to over 190,000 deaths. The primary treatment for ARDS involves mechanical ventilation alone or together with treatments directed at the underlying cause of the disorder (e.g., antibiotics, steroids). Supportive strategies, such as fluid management, sedation interruption, and early mobilization are typically used as well. ARDS is associated with a death rate between about 35% and 50%. Patients that survive ARDS have an increased risk of lower quality of life, pulmonary-disease specific health related quality of life, persistent cognitive impairment, and/or physical and psychological dysfunction. Examples of residual impairment of pulmonary mechanics and injury to the lung following ARDS include mild restriction, obstruction, impairment of the diffusing capacity of carbon monoxide, or gas exchange abnormalities with exercise.

Despite adaptation of lung protective ventilation strategy's Acute Respiratory Distress Syndrome (ARDS) still has poor long-term survival [10] and QOL [11], with mortality rates as high as 40% [12]. It is estimated that over 20% of ventilator dependent patients will be affected by this disease during any hospitalization [25] [26]. Up to 200,000 individuals sustain this illness annually in the United States alone, with 75% of these patients having severe pathology [12]. In addition, these dismal outcomes transcend to higher associated healthcare costs, poor functional status and excessive mental health issues [10] [11]. Currently, the management of ARDS lacks viable options for therapeutic intervention with supportive measures being the mainstay of treatment [27].

ARDS is defined as the acute onset of respiratory failure with bilateral infiltrates on chest radiograph, hypoxemia as defined by a (ratio of the partial pressure of arterial oxygen to the fraction of inspired oxygen) PaO₂/FiO₂ ratio ≤200 mmHg, and no evidence of left atrial hypertension or a pulmonary capillary pressure <18 mmHg [28] [29]. The Berlin Definition provides for 3 main (mild, moderate, and severe) classifications of severity based on PA/F102 ratio and associated hypoxia [28] [27]. Mortality is intimately related to these severity classifications as well as median duration on ventilatory support significantly increasing with each stage [27] [28]. Since ARDS was first described, its management has evolved, however today ventilatory support is the mainstay of therapy with the main objective being to maintain adequate blood oxygenation while avoiding oxygen toxicity [27]. This involves titration of F102 (fraction of inspired oxygen) as well as the use of supplemental PEEP (positive end-expiratory pressure) in what is termed “Lung Protective Ventilatory Strategy” [27]. The strategy entails the use of smaller tidal volumes (VT), at 5-7 ml/kg to avoid volutrauma/barotrauma [27]. PEEP assists in this regard and increases alveolar distention and recruitment, limiting repetitive injury caused by atelectasis [27]. The ARDSnet trial, found that the use of low tidal volume ventilation reduces mortality by as much as 22% [30] [27]. Additionally, low tidal volume settings reduced the risk of end organ failure and days on mechanical ventilation [27] [30]. However, a meta-analysis later found no associated clinical benefit with low tidal volume strategies with possibly an increased mortality risk with this strategy [31].

In addition, to low tidal volume strategies other treatment modalities have been utilized in ARDS treatment with mixed results [27]. One of these regimens involves the use of prone positioning. The hypothesis behind this treatment is to allow for redistribution of dependent lung infiltrates as they are typically non-uniform [27]. The use of prone positioning has had some evidence suggesting that it may enhance oxygenation by relieving atelectasis and improving perfusion and ventilation mismatches [27]. Studies have demonstrated that proning successfully recruits atelectatic lung units, improving oxygenation with some evidence supporting improvement in mortality rates [32] [27]. Another cornerstone of management is related to limiting overzealous fluid resuscitation with negative fluid balance being most advantageous in the first few days from presentation[27]. However, despite improved oxygenation this again was associated with no statistically significant difference in 60-day mortality [27]. Additional, adjunct therapies all of which have mixed consensus/benefit include: inhaled NO (nitric oxide), inhaled prostaglandin E1, extracorporeal membrane oxygen (ECMO) as well as intravenous steroid administration[33] [34] [27].

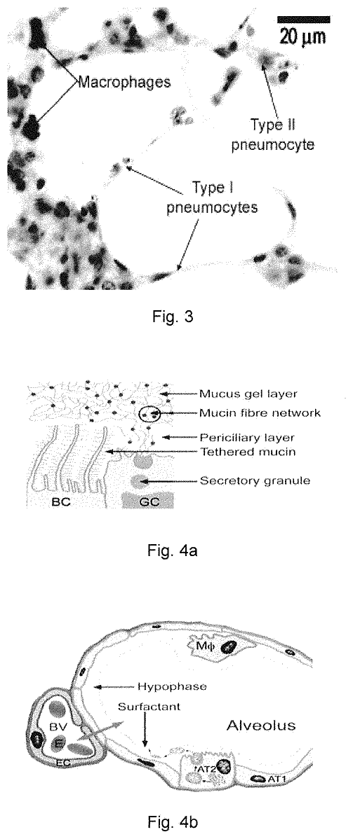

To highlight the pathology rooted in ARDS the anatomic and mechanical organization of the respiratory tract is paramount. The respiratory tract is organized into generations based on its total surface area [35]. As the airway progresses from the trachea (1^{st} generation) to its deepest structure the alveoli (23^{rd} generation), its surface area decreases rapidly from 2.5 cm² to 0.8 m\hat{ }2 (8000 cm²) respectively [36] [37] [35]. The respiratory tree can be subdivided into a conducting zone, consisting of the trachea and bronchi, and the respiratory zone containing the bronchioles and alveoli [35] [36]. The trachea conducts air from the oropharynx towards the lungs via the bronchi, which subsequently distributes air to bilateral lung bronchioles and alveolar sacs [35] [36]. Though the respiratory zone is made of two main components the alveoli are responsible for gas exchange [38] [39] [35]. There are 3 main cell types that comprise this functional alveolar unit, type 1 pneumocytes, type 2 pneumocytes and alveolar macrophages[39]. Type 1 pneumocytes comprise 95% of the alveolar surface area, are composed of thin single layer squamous epithelial cells and actively participate in gas exchange [39]. Type 2 pneumocytes are cuboidal cells with small villous projections serving as regenerative stem cells and produce surfactant, a substance that maintains alveolar patency by reducing alveolar surface tension [39]. Lastly, the alveolar macrophages are resident cells involved in clearing pathogens and alveolar debris FIG. 3 demonstrates normal alveolar microscopic structure [39].

These resident cells are all supported by an extracellular matrix (ECM) network. The ECM consists of an alveolar basement membrane which is composed of type IV and V collagen and functions to separate the alveolar epithelium from its underlying endothelial structures [40, 41]. The interphase between these comprise the alveolar-capillary barrier which is involved in gas exchange and are composed predominantly of Type I and III collagen [40] [41]. The fibrillar collagens (types I, II, III, V and XI) also contribute to the architectural organization and possess tensile strength but poor elasticity [42] [43]. In relation, elastic fibers form a delicate lattice mesh throughout the lung, are highly concentrated in areas of stress such as the areas of alveolar opening and junctions, and provide the lung with necessary compliance [44]. These elastic fibers are composed of elastin, fibrillin and fibulin, all of which are mechanically connected to ECM collagen [42] [43, 45]. The ECM framework of elastin and collagen, influence pulmonary mechanical organization of which can best describe the alveolar structure as polygonal structures adjacently tethered in an interdependent fashion [43] [46] [44, 47]. This polygonal scaffold is lined extensively with collagen fibers allowing alveoli to influence opening and closure of adjacent alveoli based on their respective patency [48] [46]. For instance, if a central alveolar unit were to collapse it would exert tethering effects on adjacent alveoli to maintain patency, likewise upon opening of a collapsed alveoli, this opening alveoli would exert stress on adjacent units to open as well [46]. This interdependence in conjunction with pulmonary surfactant maintain alveolar stability [46].

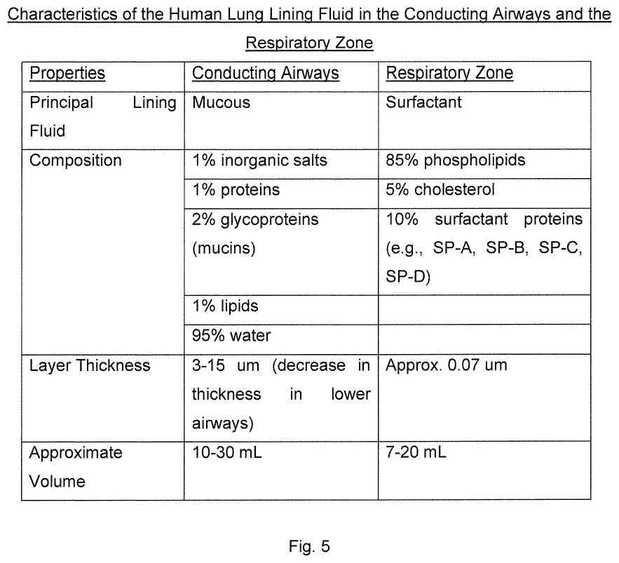

Additionally, the lung regions facing the air surface are lined by a thin film liquid called lung lining fluid (LLF) and maintains inherent respiratory function, as well as lubricating, and protecting its underlying epithelium [49]. The LLF is continuous throughout the respiratory tree, but has different compositions based on location [49]. The conducting airways for example, are lined by an airway surface liquid (ASL), a mucus gel-aqueous complex of ˜5-100 um depth functioning to trap debris and expel it from the respiratory tract [49]. The ASL complex is mostly composed of mucin glycoproteins and proteoglycans [50]. Generally, inhaled particles with <5-um diameter bypass the respiratory defenses of the conducting airways and can become trapped in the fluid lining the alveoli [49]. In comparison, the alveoli are lined by an alveolar subphase fluid (AVSF) and pulmonary surfactant with an approximate depth of 0.1-0.2 um [49]. Pulmonary surfactant as mentioned previously, is synthesized and secreted by type II alveolar epithelial cells, and is composed of phospholipids (80%), cholesterol (10%), and proteins (10%); with four specific surfactant proteins (SP) identified [51]. This interphase influences alveolar patency via surface tension modulation and air gas exchange; with its composition related to solute and fluid balance mediated by alveolar epithelial cells [49]. FIGS. 4a and 4b depict the organizational structure of the alveolar and lung lining fluid, and FIG. 5 demonstrates a comparison of their regional characteristics[52] [53]:

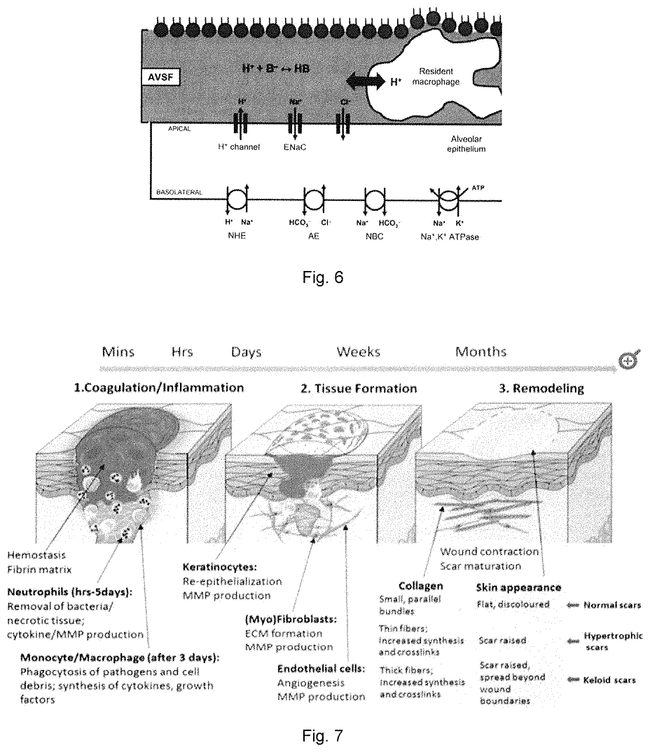

Type II alveolar also participate in active NaCl (sodium chloride) uptake, with Na+(sodium) influx occurring through apical Na+ channels (ENaC) in response to an electrochemical gradients created by basolateral Na+,K+-ATPase (sodium-potassium pump), while type I alveolar cells have been demonstrated to participate in both active and passive solute water transport [54] [49] [55]. The osmotic gradient generated by these cells leads to the reabsorption of water from AVSF [49]. These cellular transporters influence AVSF pH through the use of basolateral membrane Na+/H+ exchanger (sodium-hydrogen exchanger) [56], Cl/HCO3 (chloride and bicarbonate) exchanger [57], and Na+/HCO3 (sodium-bicarbonate) [58] cotransporters as well as apical H+ channels [59]. Relative activity of these ion channels influences local host immune function and AVSF fluidity, with a mean pH reported to be 6.9 [59], which is relatively stable due to these built in buffer systems [59] [49]. FIG. 6 depicts the organization of these various transporters involved in maintaining the AVSF [49].

### Extracellular Matrix and Injury

Pulmonary hemostasis is constantly in a state of flux, having to adapt to various insults to maintain adequate function. When injury does occur normal wound healing requires sequential ECM degradation and resorption[60]. These sequential steps and related timeline are depicted in FIG. 7 [60].

The process begins with hemostatic plug creation dictated by platelet infiltration and degranulation, releasing potent chemoattractant factors for inflammatory cells and simultaneous activation of the coagulation cascade [61, 62]. These include various chemokines, thrombin, transforming growth factor-beta (TGF-B), platelet-derived growth factor (PDGF), and vascular endothelial growth factor (VEGF) [61] [63]. Thrombin assists in this early clot formation and further propagates the release of pro-inflammatory factors including: interleukin-6 (IL-6), and IL-8 [60, 61, 64] [65]. These early inflammatory cytokines also activate complement factors (C3 and C5), and induce migration of neutrophils and macrophages transitioning the local wound environment to the inflammatory phase in a process that is necessary for proper healing[64] [61] [60] [65]. Neutrophils then secrete various of proteases and generate reactive oxygen species (ROS) to ward off pathogens and direct further recruitment of macrophages[66]. It should be noted that the presence of macrophages is necessary for proper wound healing while neutrophil presence is not, with prolonged neutrophil involvement actually predisposing to chronic non-healing wound development[66].

After 2 to 3 days the proliferative phase ensues and is characterized by angiogenesis and fibroblastic production of an ECM scaffold called granulation tissue which consists of procollagen, elastin, proteoglycans, and hyaluronic acid (HA) [60]. The recruited cells assist in the initial synthesis of this granulation tissue which allows ingrowth of blood vessels providing nutritional support and adequate oxygenation, creating a preliminary scar with a disorganized ECM framework [60]. This ECM scaffold is dynamic and modulates the wound healing process, assisting in maintaining stem cell lineage for regeneration and will undergo subsequent crosslinking and reorganization during the remodeling phase [67] [60]. During this process collagen remodeled and its relative expression dictates the wounds natural progression [60]. This remodeling of which involves early collagen degradation liberates sequestered growth factors, relaxes cellular interactions allowing for cellular migration and tissue regrowth [60]. Thus ECM and collagen remodeling serves as a reservoir for potent growth factor signals, promotes neovascularization, wound re-epithelialization, and regulates cell to cell and cell to matrix signaling in a process that continues for up to a year [67] [68] [69]. Additionally, these ECM derived byproducts interact with the various aforementioned factors throughout the wound healing cascade including platelet-derived growth factor (PDGF), transforming growth factor-beta (TGF-B), vascular endothelial growth factor (VEGF), and fibroblast growth factor-2 (FGF-2) [63]. In brief, PDGF is typically released by platelets early in the wound healing cascade and serves as a chemoattractant for fibroblasts, increasing collagen deposition into the ECM matrix [70] [63], while FGF-2 induces fibroblasts and endothelial cell growth when complexed with heparin [71] [63]. TGF-B also stimulates the synthesis of collagen and fibronectin, reduces the proteolytic degradation of ECM components and modulates TIMP expression, ultimately limiting collagenase stimulation, while VEGF is critical to neovascular growth [72] [63] [73]. Ultimately, these factors contribute to micro-environment collagen deposition and control cellular lineage expression during ECM remodeling [63]. As this process continues early type III collagen deposition is subsequently replaced with type I collagen [68] [60] [74]. This scar replacement coincides with the remodeling phase and incorporates vascular maturation with subsequent finalization of re-epithelialization[68] [60] [74].

As alluded to, ECM composition is intimately involved with the wound healing process, making it essential in maintaining biomechanical and structural support. Its dynamic role during wound remodeling is critical to preserving normal tissue function and architecture [47] [75]. Thus, the composition of ECM ultimately determines the biomechanical and physiological properties of that specific organ, including tensile strength, elasticity and compliance [47]. ECM is composed primarily of collagen and non-collagenous proteins including elastin, proteoglycan, glycoproteins, fibronectin, as well as laminin, amongst others [41]. As the most abundant component of ECM however, levels of collagen expression are not only a key aspect of maintaining tissue function and organization but are also of importance when derangements in its remodeling occur [60, 76].

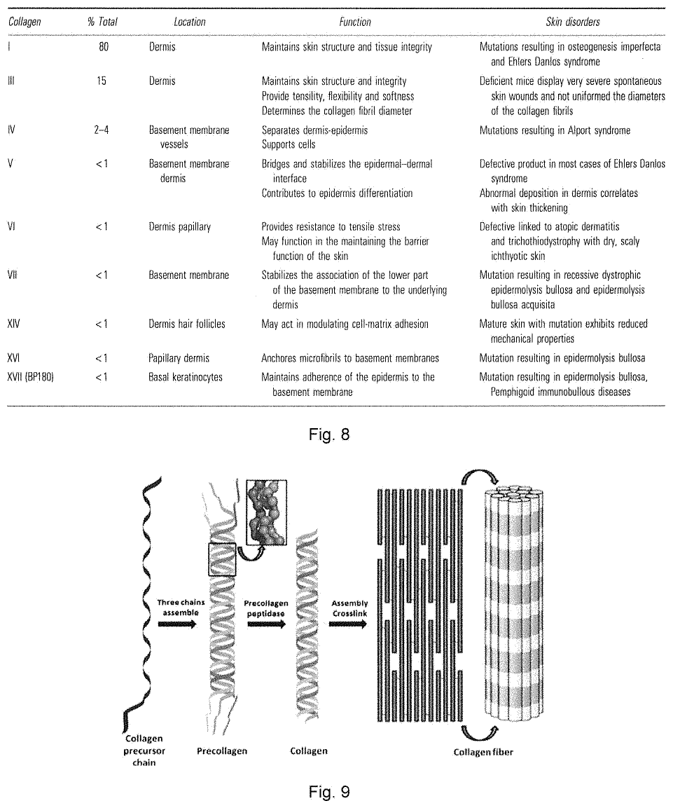

Collagens are a family of peptides whom possess a triple poly-peptide alpha helix configuration held together by interchain hydrogen bonds [60]. It has over 28 different subtypes with distributions largely dictated by its microenvironment and tissue/organ location [60, 77]. Though there are differences amongst the types of collagen subtypes they have a relatively conserved repeating sequence of Glycine (Gly)-X-Y triplets, where X and Y are frequently proline and/or hydroxyproline [77] [78]. These polypeptide chains are flanked by nonhelical regions of which are characteristically found in all procollagens [78] [77]. FIG. 8 lists the various types of collagens, their function as well as the relative distribution throughout the body [60].

Collagen can be sub-classified as fibrillar and/or fibril associated collagens [60]. Fibril associated collagens possess an interrupted triple helix conformation and include types XII, XIV, XVI, and VI [79] [60]. These types of collagens spontaneously aggregate after processing of their pro-collagen forms into an ordered fibrillar structure [79] [60]. The fibrillar (fibril forming collagen or interstitial) collagens include types I, II, III, V and XI, and are synthesized primarily by fibroblasts as pro-collagen peptides which contain N-(amino) and C-(carboxy-) terminal regions [43] [79] [60]. They maintain a helical structure, consisting of three polypeptide chains as well, with the three chains uniting to form a right-handed super helix[79].

Collagen synthesis occurs both intracellularly and extracellularly, and involves post-translational modification of pro-collagen peptides through a series of enzymatic reactions with subsequent cross-linking [80] [60]. This process begins with cleavage of its N-terminal signal peptide, hydroxylation of lysine and proline residues, followed by glycosylation of lysine associated hydroxyl groups with galactose and glucose [80]. Once this occurs three of these hydroxylated and glycosylated left-handed pro-alpha-chain helixes assemble into a right-handed triple helix configuration, creating the pro-collagen molecule [80]. The pro-collagen is then transported to the Golgi-apparatus for additional modifications and then assembled for secretion to the extracellular space [80]. The pro-collagen molecule is then cleaved by specific peptidases making tropo-collagen [80]. The tropocollagen lysine and hydroxylysine residues, are subsequently cross-linked by lysyl oxidase, creating covalent bonds between tropo-collagen molecules to form the collagen fiber [80] [60]. FIG. 9 depicts the assembly of collagen and its structure [60]:

The final collagen product is then incorporated into the ECM in an interlaced basketweave-like arrangement, making collagen a major influencer of ECM architecture and function [48] [80] [60]. Additionally, the covalently crosslinked bonding stabilizes their structure and explains the inherent tensile strength and the resilience they possess[48] [80] [60]. Collagen can then undergo various organizational templates, varying from random orientation (lung tissue, cartilage) or quasi-structured networks as seen in tendon [79] [81]. In relation, in compliant tissues like skin and lung for instance, collagen within the ECM is additionally interwoven with a protein called elastin [44] [60]. Elastin is a deformable protein that provides flexibility, allowing tissues to stretch and subsequently recoil, which also alters overall ECM structure [44] [60].

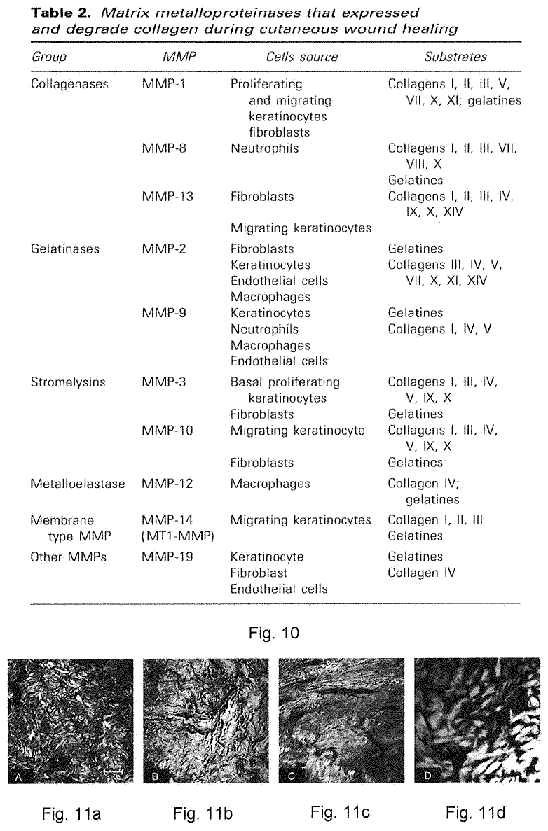

Appropriate ECM remodeling requires a balance in relative collagen expression, of which is determined by two main classes of enzymes, tissue inhibitors of matrix metalloproteinases (TIMP) and matrix metalloproteinases (MMP's) [82] [60]. FIG. 10 lists the various classes of MMP's and their respective function[60].

MMPs are a class of enzymes that catalyze the hydrolysis of ECM components including collagen [82] [83]. They are zinc and calcium dependent endopeptidases with multifunctional domains with a core structure consisting of a pro-peptide, a catalytic metalloproteinase domain, a linker peptide (hinge region), and a hemopexin domain [84] [85]. The pro-peptide domain contains a cysteine sulfhydryl motif that chelates the active site zinc (Zn2+) functioning to keep the enzyme in its inactive pro-MMP zymogen form [84]. During activation of the enzyme this cysteine is cleaved and the pro-domain is detached often by various proteolytic enzymes [84]. The C-terminal hemopexin domain consists of two active modules with a deep zinc (ZN2+) dependent active site cleft, which is highly conserved and is bound by three histidine residues from a conserved sequence of HEXXHXXGXXH (H=Histidine, E=Glutamic acid, G=Glycine, X=any amino acid) [85]. This hemopexin domain determines substrate specificity unique to each MMP and is determined by a hydrophobic pocket of variable depth (S1, S2, Sn . . . etc) [84]. Additionally, this hydrophobic pocket is stabilized by two to three calcium ions, as well as a conserved glutamate and methionine residue [86] [84]. The substrate specificity of this motif is exemplified by its interactions with distinct collagen types, for example fibrillar collagens are only degraded by MMP-1, MMP-8, and MMP-13 [60].

To accomplish collagen digestion MMP's unwind triple-helical collagen cleaving the three alpha chains of the helical structure of type I, II and III collagens at particular recognition sequences Glutamine (Gln)/Leucine (Leu)-Glycine (Gly) #Isoleucine (Ile)/Leucine Leu) Alanine (Ala)/Leucine (Leu) with cleavage performed after the Gly residue (# indicates the bond cleaved) [86] [84]. During this MMP-substrate interaction, Zn2+ is positioned towards the substrate's carbonyl oxygen atom, with one oxygen atom from the MMP glutamate-bound to water, and the three-conserved histidines [86] [84]. A nucleophilic attack on the substrate is then initiated by the Zn2+-bound to water initiating the breakdown of the substrate molecule [86] [84]. The unwound collagen is then digested into specific fragment sizes, with type I, II, and III collagens typically portioned into three-fourth and one-fourth fragments [84, 86].

After this initial digestion, the remaining collagen structure is subsequently degraded by a related MMP class called gelatinases, specifically MMP-2 and MMP-9 [60]. Of note the hemopexin domain of these MMPs seems to be essential for unwinding collagen's triple helical structure, while the catalytic domain retains ability to denature non collagenous and unwound collagen byproducts [84] [60] [86]. It should be mentioned that some of these MMPs have also been implicated in elastin degradation and include MMP-2, MMP-7, MMP-9 and MMP-12 [87, 88]. Typically, the hemopexin domain is essential for cleaving native fibrillar collagen while the catalytic domains are responsible for cleaving non-collagen substrates, however this is not consistent across all subtypes [86] [84]. The tissue matrix metalloproteinase inhibitors (TIMPs) are enzymes with 4 specific subclasses, whom serve to counteract MMP activity through direct interaction with the enzymes active site [60]. The balance between local expression of these two enzyme classes greatly influences ECM remodeling and ultimately determines the final characteristic of the repaired tissue [60].

### Pathology of Extracellular Matrix Remodeling

It has been demonstrated that any disruption or delay in wound remodeling can cause abnormal re-epithelialization, abnormal scar formation, ultimately with less vascularity compared to native pre-injury tissue [76]. These pathological states are often due to a skewed inflammatory response, with evidence suggesting that inhibition of excessive inflammation can limit or avoid these states[89] [60]. For instance reduced inflammation via activated protein C administration in chronic adult wounds stimulates angiogenesis as well as dermal and epidermal regrowth [89] [60]. Additionally, fetal wounds are characterized by a less robust inflammatory response, the opposite of what is observed in adults, often resulting in relatively scarless wound healing [90] [60]. Thus the extent of initial inflammation after injury has profound effects on proper wound healing, therefore this response must shift from its initial M1 (pro-inflammatory macrophage state) to an M2 phenotype (anti-inflammatory macrophage state) to limit abnormal tissue remodeling [91] [92].

In contrast numerous pathologies are observed if this inflammation is not curtailed, intimately influencing ECM remodeling and collagen turnover. To begin, hypertrophic scars are characterized by raised, erythematous lesions, occurring in regions of high tension and tend to not go past the margin of the scar[93] [94] [60]. Histologically, hypertrophic scars demonstrate excess type III collagen oriented parallel to the epidermal surface with abundant nodules containing myofibroblasts and large extracellular collagen filaments [94] [95]. Ghahary et al. found that expression of collagenase was significantly reduced hypertrophic scars (51±7% vs 100±11%; n=5; p<0.05) and resulted in accumulation of extracellular matrix overexpression of fibronectin as well as type I and type III pro-collagen [96]. This local deficiency of native collagenase has also been corroborated by reduced collagenase gene expression in hypertrophic scar fibroblasts [97]. A related dermal condition, keloid scars, tend to infiltrate into surrounding tissues past the margin of scar formation and histologically are composed of disorganized type I and III hypocellular collagen deposition[93] [94]. Studies have attributed these changes to a three times greater collagen synthesis at all remodeling phases during keloid formation, with significant increase in relative collagen synthesis compared to normal scar formation (679+/−109, vs 224+/−54, p<0.001) [98] [95]. ECM pathology is also observed in conditions like Dupuytren's contracture, which is a condition characterized by progressively abnormal thickening of the skin usually at the base of fingers or joints of the hand[99] [100] [101]. This condition is caused by a prolonged inflammatory state, leading to excessive myofibroblast remodeling of type I collagen and excessive type III collagen deposition[99] [100] [101]. Histologically, one observes extensive interconnected myofibroblasts tethered to ECM fibronectin and extracellular fibrils composed of predominantly type III collagen[99] [100] [101]. This ultimately causes shortening of fascial bands leaving tissue inelastic, with tendon-like cords and digital flexion contractures [99] [100] [101] [102]. Additionally, cosmetic conditions such as cellulite are also related to ECM changes and is characterized by excessive fibrous septa creating a dimpled appearance of the skin [103] [104]. The mechanism behind this condition is structural and biomechanical ECM collagen imbalances at the subdermal junction, leading to irregular subcutaneous fat extrusion and contour [103] [104].

FIGS. 11a-11d highlight the abnormal collagen deposition observed in keyloid and hypertrophic scars at the microscopic level[93]. FIG. 10a illustrates normal skin, FIG. 10b is a normotrophic scar, FIG. 10c is a hypertrophic scar, and FIG. 10d is a keloidal scar.

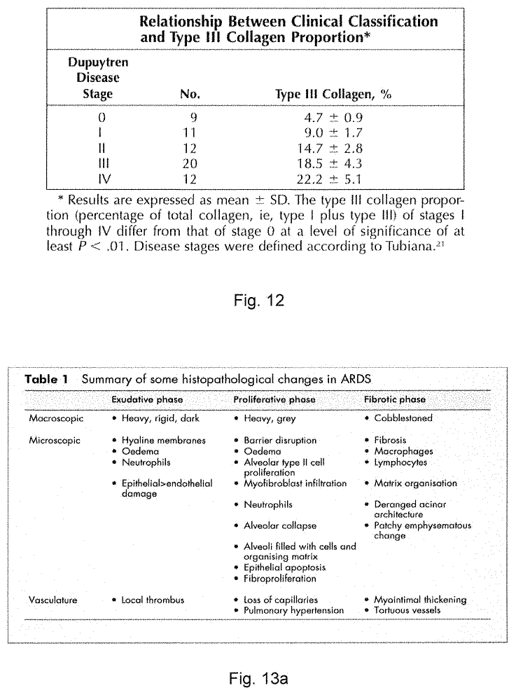

FIG. 12 is a table depicting the changes in collagen content observed in Dupuytren's Contracture [101].

ECM collagen homeostasis is also intimately related to pulmonary pathology. For instance, emphysema is a condition caused by inflammation and remodeling of the distal airways and lung parenchyma that manifests as loss of surface area for gas exchange [105] [106]. During emphysema this remodeling damages extracellular matrix causing a reduction in elastic recoil and an overly compliant lung [105] [107]. Numerous clinical studies have attributed this disease process to aberrant pulmonary expression of various MMP's including: MMP-1, MMP-2, MMP-8, MMP-9, MMP-12 and MMP-14 [108] [109] [110] [111] [112]. Ultimately these expressive changes lead to an imbalance in ECM collagen turnover [107]. Furthermore it has been suggested that emphysematous lung scaffold can fracture more readily at normal breathing pressures and stress, suggesting that post-ECM remodeling collagen content is overall weaker than its baseline form [113] [48]. Similar ECM imbalances have also been observed during the development of idiopathic pulmonary fibrosis (IPF) [114] [16]. The subsequent cascade in this condition leads to ECM architectural distortion and is largely due to overexpression of TIMP-1 and TIMP-2 with reduced MMP expression in densely fibrotic interstitial regions [16]. In relation, ECM remodeling in IPF has demonstrated increased total collagen content with an associated increased stiffness in native lung parenchyma [115] [48]. Montano et al., attributed these pathological changes due to reduced collagenolytic activity, lower native MMP expression, and relatively higher TIMP activity [114]. Histologically, these changes lead to thickened alveolar septa, condensed fibrotic ECM-conglomerates within the interstitial space and alveoli [116] [117] [42]. The microscopic effects of these fibrotic changes contribute to diminished effective gas exchange, worsening lung function and respiratory failure [42] [116]. These changes have also been corroborated in a bleomycin animal model, with increased rates of fibrillar type I, III and V collagen synthesis during the later phase of remodeling [118]. Pathological ECM remodeling are due to the continued prolonged presence of non-viable debris which propagates pro-inflammatory cytokines, worsening fibrosis and scarring [119]. This in combination with the presence of limited microcapillary growth, limits monocyte migration, and creates a prolonged M1 (inflammatory macrophage) response limiting proper healing [92]. The changes in both epithelial and lung disease again highlight the influence ECM remodeling can have on end organ organization and function.

### Extracellular Matrix Remodeling and Acute Respiratory Distress Syndrome (ARDS)

The aforementioned importance of ECM homeostasis and its remodeling are intimately related to the pathogenesis of ARDS and the subsequently deranged pulmonary mechanics [41]. After alveolar damage the ECM undergoes remodeling in 3 well characterized phases, similar to dermal pathology, and include: exudative, proliferative and fibrotic stages[120] [7]. However, despite these defined phases there is considerable overlap [7], therefore these phases can be classified as early and late (organizing) in relation to the time from initial presentation, to a cut off of approximately 7 to 10 days[120] [121]. The early stage of ARDS includes the exudative phase which begins within 24 hours and can last up to a week [7]. Histologically, it is characterized by diffuse alveolar damage, edema, hyaline membrane formation and alveolar epithelial necrosis lined by collagen and fibrin [7]. The reactive inflammation leads to accumulation of neutrophils, leukocytes, a altered endothelial and epithelial barrier function, [122]. This subsequent alveolar damage and the continual mechanical stress in accommodating tidal volumes [48] [123], influences the architectural load bearing ability of the alveoli with collagen content arguably being the most influential factor [124] [48]. This excessive mechanical stress is a prominent factor in exacerbating ECM deposition which can lead to cellular epithelial-to-mesenchymal transition (EMT) into myofibroblasts whom worsen tissue stiffness, limit neovascularization and fibrotic architectural distortion [47].

Collagen content is a known factor in lung tissue compliance and elasticity influencing compliance in a nonlinear fashions with increasing volumes [125]. Animal models have found that excessive collagen deposition increases exponentially with the severity of lung injury and influences fibroelastosis [126]. This collagen deposition is not only in excess but also results in abnormal collagen fiber organization, worsening pulmonary resistance and compliance [115]. A study by Armstrong et al, found that ARDS patients have a significant imbalance in collagen turnover; with an early shift towards collagen synthesis in comparison to at risk patients [127]. The observed excessive collagen synthesis in this study also correlated with severity of lung injury scores and predicted progression to ARDS vs those subjects who were at risk (median values, 124.9 and 95.0 ng/ml versus 38.0 ng/ml, respectively, p<0.0005) [127]. This imbalance in ECM turnover has been corroborated by additional evidence and again occurs quite early in the course of ARDS, with elevated collagen deposition seen within 24 hours [15] [13]. Elevation in pro-collagen levels also significantly correlated with fibroblastic mitogenic activity with this effect lasting past 7 days [15]. Furthermore, these early changes influence recovery and outcomes with the relative risk (RR) for death increased in patients with elevated procollagen III levels of 1.75 U/mL or more obtained on day 3 (RR, 2.4; 95% Cl, 1.3 to 4.3), day 7 (RR, 2.7; Cl, 1.4 to 5.4), and day 14 (RR, 2.7; Cl, 1.1 to 6.3) [13].

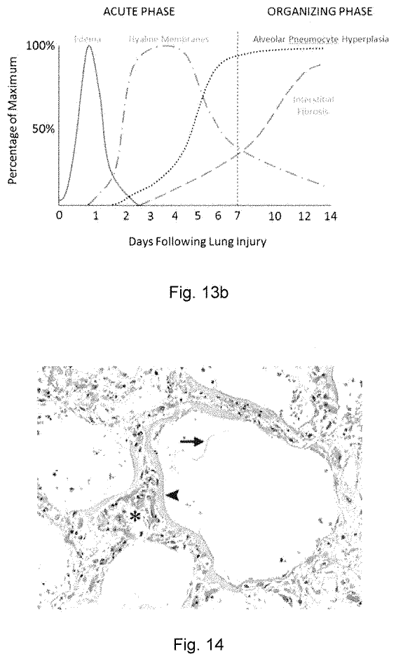

FIGS. 13a and 13b depict the histological changes associated with each of the described phases of ARDS and the time course of the relative phases of its pathogenesis[120] [7].

After the exudative phase, the proliferative phase ensues, which causes progressive fibrosis and replacement of the thin alveolar interphase with a thickened scarred ECM [7]. This corresponds with the commencement of the late (organizing) phase of ARDS, which again begins approximately 7-10 day's from presentation and is characterized by disordered remodeling [120, 128] [121]. During this transition the obstructed alveolar lumen is remodeled converting initial hyaline membranes and cellular debris into fibrotic tissue secondary to intense proliferation of type-2 pneumocytes and fibroblasts [128] [129]. This proliferative phase reaches its peak at 2 to 3 weeks and can progress to fibrotic replacement of lung architecture [121] [128]. This leads to excessive accumulation of type I and III collagen at the alveolar interphase, similar to observed changes in IPF, impairing diffusion of gases, damaging the normal mechanical properties of the lung, propagating a ventilatory mismatch and associated reduction in lung compliance [41] [7]. Studies have correlated thicker collagen fiber diameter, as well as the amount of relative collagen crosslinking during remodeling, to the increased stiffness of the ECM architecture as the main culprit behind the reduced compliance seen in ARDS [48, 130]. Planus et al, confirmed the importance of MMP/TIMP balance during ECM lung remodeling and its influence on the lung cytoskeleton; with improved alveolar compliance directly proportional to MMP expression [17]. This local MMP expression also correlated with a two-fold accelerated alveolar healing time and enhanced type 2 pneumocyte migration as well [17]. An in-vivo human study has attributed these changes to skewed MMP/TIMP levels with significantly increased TIMP-1 and TIMP-2 expression and a virtual lack of MMP's observed in dense fibrotic regions of the lung and in hyaline membranes[16]. In-vitro animal studies have shown that this ARDS fibrotic remodeling can be effectively modulated with supplemental exogenous collagenase; with its application improving degradation of pathological collagen, expediting the repair process, accelerating re-epithelialization and enhancing migration of type 2 pneumocytes [131].

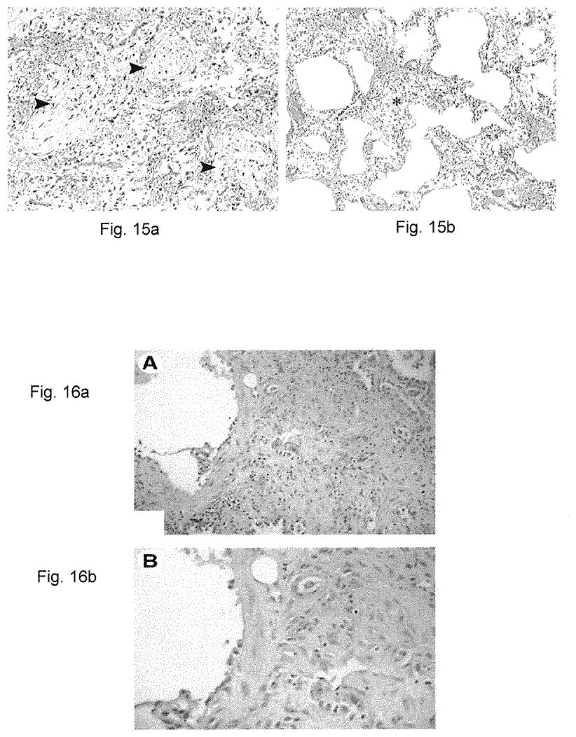

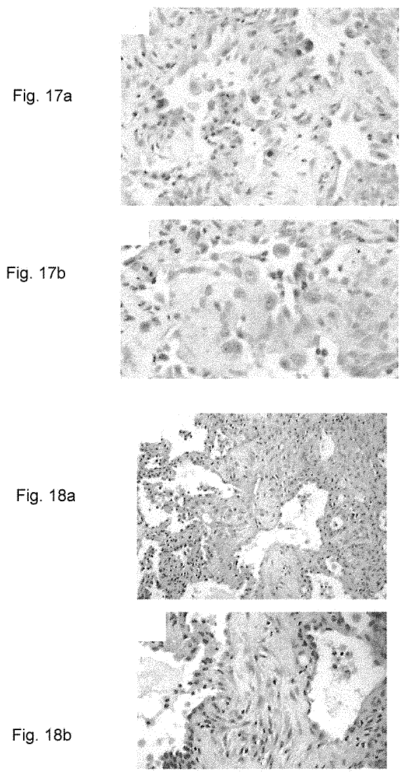

FIGS. 14-17 highlight the histological changes observed during the course of ARDS [132] [120]. Specifically, FIG. 14 is a photomicrograph of acute phase DAD (original magnification ×200 H-E stain), showing characteristic hyaline membranes at the arrows and alveolar wall edema in acute phase DAD. Capillary leak has resulted in amorphous eosinophilic edema fluid in the alveolar spaces. FIGS. 15a and 15b are photomicrographs (original magnifications ×320 (FIG. 15a) and ×100 (FIG. 15b) in H-E stain in the same patient showing organizing fibroblastic tissue as plugs within the alveolar spaces (arrows in FIG. 15a and diffusely involving the interstitium (stars in FIG. 15b). FIGS. 16a and 16b illustrate diffuse alveolar damage in the proliferative phase. FIGS. 17a and 17b illustrate diffuse alveolar damage with significant cytologic atypia. FIGS. 18a and 18b illustrate diffuse alveolar damage in the early proliferative phase. [132]

Collagen imbalance not only influences physiologic and biomechanical function, but alters outcomes, with the amount of lung collagen deposition being most influential on ARDS lung recovery [133] [14] [15]. In relation, elevated TIMP levels are associated with significantly higher risk of mortality (10,373±1,602 ng/ml vs 4,737±972; p=0.0067) [9]. In relation, higher MMP/TIMP expression ratio in late phase BAL (broncho-alveolar lavage) samples is associated with improved survival (112±77 vs Non-Survivors: 0.78±0.24; p<0.05) [9]. Additionally, in-vivo studies found significantly reduced collagen content and in those with higher amounts of MMP-2 expression in (BAL) samples [8]. This same study also found significantly improved mortality in ARDS patients in those with greater MMP-2 expression and found pan inhibition of native MMPS to significantly worsened mortality and hampered alveolar healing [8]. The progression to pulmonary fibrosis has been demonstrated to intimately being related to total lung collagen content [133] [134]. The correlation in excessive collagen deposition and prognosis [14], also influences those receiving extra-corporeal membrane oxygenation (ECMO), a measure often utilized to supplement ARDS lung recovery, with type III procollagen elevation having significantly greater odds of mortality (odds ratio=1.37, 95% Cl: 1.10-1.89, p=0.017; receiver operating characteristic (ROC)/area under the curve (AUC)=0.87, 95% Cl 0.69-1.00, p=0.0029)[135]. These evident changes again highlight the influence ECM collagen imbalances can have on ARDS recovery.

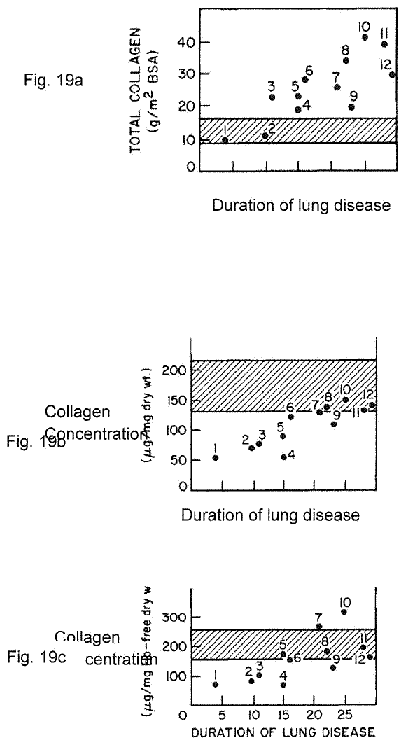

FIGS. 19a-c highlight the results of a related study assessing the changes in collagen content observed in ARDS patients [133]. Total collagen (gram per m² body surface area) and collage concentration in the lungs of patients at various times after the onset of acute respiratory failure (RAF). Each point is the mean for a large number of postmortem lung samples. The shaded areas encompass the mean values for the 9 normal lungs. The numbers associate with the closed circles identify the patients with ARF. Kendall's rank correlation coefficient was calculated as a measure of the association between duration of the lung disease and the concentration of collagen in the lung. Kendall's coefficients were 0.65, 0.71, and 0.50 for the data on total collagen, collagen concentration per mg dry weight, and collagen concentration per mg hemoglobin-free dry weight, respectively. The 3 coefficients were significantly larger than zero. [133]

To better understand its progression numerous markers of injury have been implicated during the course ARDS with mixed utility. Some of the more prominent markers include: advanced glycation end-products (sRAGE) [136], Krebs von den Lungen-6 protein (KL-6) [137], lactate dehydrogenase (LDH), vascular endothelial growth factor (VEGF [138]), surfactant protein SP-D [139], angiopoiten-2 (Ang-2) [139], von Willebrand factor (VWF) [139], as well as various interleukins, (IL-8) [121] [140]. sRAGE levels in the plasma and the bronchoalveolar fluid in animal models correlate with alveolar fluid clearance (AFC) a necessary aspect for ARDS resolution, but has had mixed consensus in mortality correlation in adults [141] [121] [136]. Another of these markers KL-6, has demonstrated diagnostic and prognostic utility for various pulmonary diseases, serving as an alveolar epithelial lining disruption marker [137]. Additionally, plasma elevations of KL-6 was higher in non-survivors than survivors, and correlated negatively with arterial oxygen tension: inspiratory oxygen fraction (PA/FIO2) indices [137]. Surfactant proteins, like SP-D for instance, are exclusively made by type 2 pneumocytes, serving as a marker for epithelial injury, with its serum elevation correlating with ARDS mortality [139]. VEGF levels in BAL samples have been found to correlate with survival with initial day zero samples being higher in those whom survived (survivors: 5.5 ng/mL (IQR: 2.3-19.7) vs non-survivors: 1.7 ng/mL (IQR: 0.0-6.4)), with same trends observed on days 5, 7 and 10, (p<0.05) [138]. TGF-B and pro-fibroblastic factor levels are also intimately related to the damage associated with ARDS and increase significantly due to continued repetitive alveolar trauma during tidal volume accommodation; ultimately worsening alveolar fibrosis, due to inhibition of essential matrix degradation enzymes including native MMPs [7] [133]. A meta-analysis has provided insight into the utility of these various markers, with elevated KL-6 (odds ratio [95% Cl], 6.1 [3.0-12.1]), lactate dehydrogenase (odds ratio [95% Cl], 5.7 [1.7-19.1]), sRAGE (odds ratio [95% Cl], 3.5 [1.7-7.2]), and VWF (odds ratio [95% Cl], 3.1 [2.0-5.2]) strongly associated with ARDS diagnosis in at risk patients [140]. This same study demonstrated that elevations in interleukin-4 (odds ratio [95% Cl], 18.0 [6.0-54.2]), interleukin-2 (odds ratio [95% Cl], 11.8 [4.3-32.2]), Ang-2 (odds ratio [95% Cl], 6.4 [1.3-30.4]), as well as KL-6(odds ratio [95% Cl], 5.1 [3.0-12.2] were most predictive with mortality during the course of ARDS [140]. In addition, IL-8 levels also correlate with prognosis, again highlighting the influence of initial inflammation on ARDS pathogenesis, ECM remodeling and outcomes [139].

### Normal Lung Ventilation and Mechanics

During respiration alveolar cellular composition, characteristics of the airways, ECM organization and the state of the AVSF/LLF all influence the ability to effectively perform respiratory gas exchange [39]. To accomplish this the alveoli require the ability to efficiently expand and recoil with the respiratory cycle [39], of which three main factors must be considered, compliance, elastance and airway resistance [142] [143]. Compliance is defined by the following equation C=ΔV/ΔP, where C is compliance, ΔV is change in lung volume, and ΔP is change in lung pressure and is the willingness of the lungs to distend [143]. Normal lung compliance is approximately in 0.2-0.3 L/cm H₂O (2-3 L/kPa (kilopascal)) [35]. The inverse of this is elastance (E˜1/C), and is the willingness to return to the resting position [143]. Resistance is defined by, R=ΔP/F, where P is pressure and F is flow, which incorporates the frictional resistance and gas flow impedance of the airway [142].

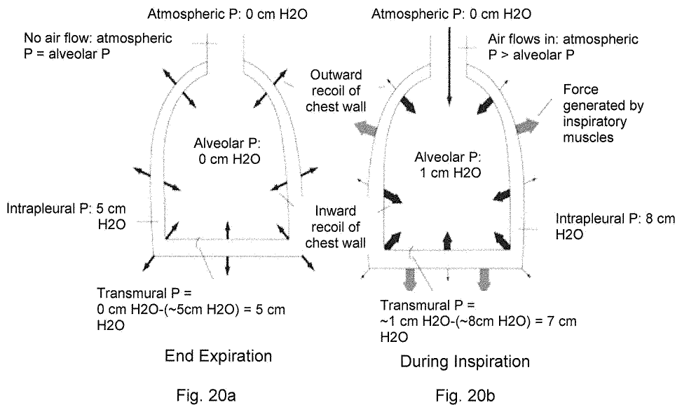

At baseline, the lung parenchyma has a tendency to collapse inward, due to alveolar elastic recoil, which is counterbalanced by outward recoil of the thoracic cavity [46]. The intrapleural pressure (Pip) is created by this alveolar-chest wall recoil interaction and is typically −3 to −5 cm H₂O at rest [46] [46]. During inspiration and expiration, the air which enters and exits the lung is called vital capacity, of which most, but not all reach the alveoli to participate in gas exchange [35]. The volume that reaches the alveoli is termed alveolar minute ventilation and is approximately 5 liters/min in normal adults [35]. The air flow that is generated during the respiratory cycle is determined by pressure gradient generation, the elastic recoil of the respiratory system and the relative resistance of the airway [46] [144]. Transmural pressure (TMP) is the pressure at a given volume required to maintain and initiate lung inflation, and is defined as the difference between alveolar (Pal) and intrapleural pressure (Pip) [35] [145]. Alveolar pressure is defined as being equal intrapleural pressure (Pip)+ alveolar elastic recoil pressure. Prior to inspiration Pal is equal to atmospheric pressure (Patm), which is conventionally denoted equal to 0 cm H2O and is the ambient pressure outside of the body [46]. To initiate inspiration the diaphragm musculature contract creating a transmural pressure (TMP) gradient causing the thoracic volume to expand and overcome airway resistance [46]. As the TMP gradient becomes more negative, Pip and Pal progressively become more negative as well, to a level below atmospheric pressure to generate air flow and distension of the alveolar sacs [46]. These pressure volume gradients during respiration are depicted in FIGS. 20a and 20b [46].

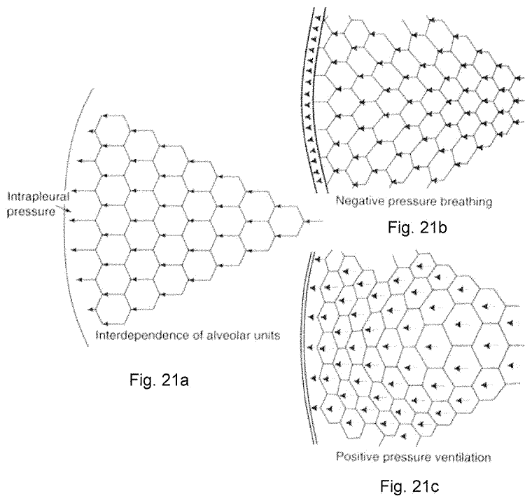

As mentioned previously, structural interdependence transmits the alveolar pressure and subsequent opening during air flow, from the central airway to the more peripheral alveoli [46]. At the end of inspiration, alveolar distention causes Pal to equilibrate with Patm once again, leading to cessation of air flow [46]. Shortly after this equilibration, expiration begins in a passive fashion causing Pip to become less negative, decreasing the transmural pressure gradient allowing the normal elastic recoil of the alveoli to return to its pre-inspiratory state [46]. The subsequent recoil leads to expiratory air flow that varies linearly with lung volume and decreases exponentially with time [46] [144]. During this process interactions between collagen-elastin ECM again allow for this mechanical cycle to occur [42] [146] [48]. The lung is constantly under baseline tensile stress throughout the ventilatory cycle as a result of the TMP gradient generation [147] [48]. The stress that is experienced by the alveolar unit is transmitted throughout the constituent cellular apparatus via the ECM scaffold and influences biomechanical function, contractility and tissue homeostasis [147]. A schematic of the alveolar interdependent scaffold is depicted in FIGS. 21a-21c [46].

This biomechanical respiratory cycle aims to accomplish diffusional gas exchange at the level of the alveoli [148]. Air that enters the respiratory tree during the inspiratory phase fills the alveoli with fresh gas that is high in oxygen (02) content and low carbon dioxide (CO2) content [148]. The polygonal alveolar walls again are supported by a very thin interstitial ECM matrix with a rich capillary network [148]. The pulmonary arterial system provides blood from the systemic circulation via the right heart that is lower in 02 and high in CO2 content [148]. The gradient of 02 and CO2 between inspired alveolar air and the pulmonary arterial circulation allows gas exchange to occur through simple passive diffusion [148]. Once the gas exchange has occurred the blood with higher 02 and lower CO2 content then flows to the pulmonary veins and into the left heart for distribution to the body [148]. The reduced alveoli volume during the expiratory phase returns gas that is lower in O2 and higher in CO2 up the bronchial tree and into the ambient environment [148]. Numerous factors can influence ventilation and gas exchange during the respiratory cycle and subsequently alter the supply of oxygen and carbon dioxide removal [148]. These factors involve: ventilation, diffusion, which includes physical diffusion across alveolar: blood barrier as well as subsequent chemical reactions (between oxygen (O2) and hemoglobin (Hb) and carbon dioxide (CO2), and lastly perfusion [148]. Diffusional conductance of inspired gas is related to the thickness of the blood:gas barrier, the overall alveolar-capillary contact surface area, as well as the weight and solubility of the gas [148]. Any disruption in these parameters including compliance, thickness of the barrier can significantly affect pulmonary ventilation and function.

### Characteristics of Clostridial Collagenases

Currently there is an MMP derived from the bacteria Clostridium Histolyticum (Ccol) that has demonstrated clinical use for ECM pathology. Ccol is available as a powder in its crude form [78] [149] [150]. Ccol is water-soluble Zn proteinase composed of two types of collagenases (type: G, ˜114 kDa and type: H, ˜110 kDa), a neutral metalloproteinase (˜35 kDa), and clostripain (˜58 kDa) a cysteine protease, with a chemical structure of C₅₀₂₈H₇₆₆₆N₁₃₀₀O₁₅₆₄S₂₁ [20]. Ccol functions at an optimal pH range of 6.3-8.8 [21], remains stable and maintains its enzymatic ability in water and saline solutions, amongst others [149] [107]. The Ccol's are considered true endopeptidases and are derived from two distinct genes of which both belong to the M9 family of MMPs [151] [78]. These genetic differences also define its enzymatic classification: col G gene, which codes for the 936 amino acid protein (collagenase type I ˜114 kDa) and col H gene which codes for the 1021 amino acid protein (collagenase type II ˜110 kDa) [151] [149] [78]. In additional at least five gelatinases have been identified in the Ccol complex, of which are responsible for denatured collagen degradation [151] [78]. Their overall molecular structure however is quite conserved and composed of two main portions: the N-terminal collagenase module and the C-terminal recruitment domain [149] [78] [150] [149]. The collagenase module possesses an activator (N-terminal) and peptidase (C-terminal) domain of which a conserved HEXXH catalytic zinc-binding motif is characteristic of the peptidase domain [149] [78]. The recruitment domain contains one to two collagen-binding domains (CBDs) as well as one to two polycystic kidney disease (PKD) like domains [149] [78]. The CBD contains two calcium ions within its cleft, necessary for stability, and assists in forming a folded beta (B)-sheet configuration [149] [78]. The PKD-like assume a V-set conformation, with its domain also containing calcium ions for stability and interdomain alignment [149] [78]. Differences amongst the various Ccol enzyme subtypes are generally rooted in the composition of the C-terminal recruitment domains and/or the zinc-binding motif sequence [149] [78].

These two classes of collagenases have unique properties, bind to unique collagen sequences and possess different specificities for native collagen degradation, in an efficient synergistic fashion [152] [78] [153] [152]. To begin, CoIG's structure is characterized by one PKD-like domain and two CBDs [150]. It contains N-terminal activator and C-terminal peptidase domains as well that form a unique saddle-shaped structure with two distinct configurations during the degradation of collagen [149] [78]. The smaller N-terminal side comprises the left saddle flap and contains an activator domain at residue (Tyrosine (Tyr)119-Aspartic acid (Asp)388) [78]. The saddle is organized as 12 parallel alpha-helices, followed by ten tandemly repeated HEAT (heat shock protein) motifs involved in protein recognition, and is flanked by the right side saddle peptidase domain [78]. The full collagenase activity is however located at residue (Tyrosine (Tyr)119-Glycine (Gly)790) and includes both the activator and peptidase domains[149]. During collagen binding and unraveling, the N-terminal activator domain and the catalytic subdomain combine to form the seat of the saddle in what has been described as a distorted four-helix bundle at residue (Tyrosine (Tyr)119-Lysine (Lys)161)[149]. In the closed state the activator HEAT motifs interact with the triple-helical collagen substrate and initiate the unwinding of the triple helix with subsequent cleavage [78] [149]. In this conformation CoIG's saddle conformation essentially compresses the collagen microfibril like a pair of pliers, leaving a single triple helix surrounded by its activator and peptidase domains [149]. Only in this state are the activator HEAT repeats able to interact with triple-helical collagen and initiate the unwinding of the triple-helix chains with subsequent cleavage [149, 150]. When the helix is completely unwound the collagenase then relaxes to the open conformation allowing other portions of the microfibril to enter the collagenase unit for subsequent unwinding and digestion [149]. CoIG method of collagen processing is described as a “chew and digest” mechanism, demonstrating processivity and substrate specificity[149]. This mechanism essentially limits the amount of viable tissue the CoIG enzyme can digest as it is shifted into its open state configuration when denatured collagen is present, as it requires N-terminally extended peptides to interact with its enzymatic motifs to obtain full activity [149, 154]. Of note the collagenase unit of CoIG can degrade collagen triple helices independently of recruitment domain assistance, however larger sized substrates may require recruitment domain activity [149] [78].

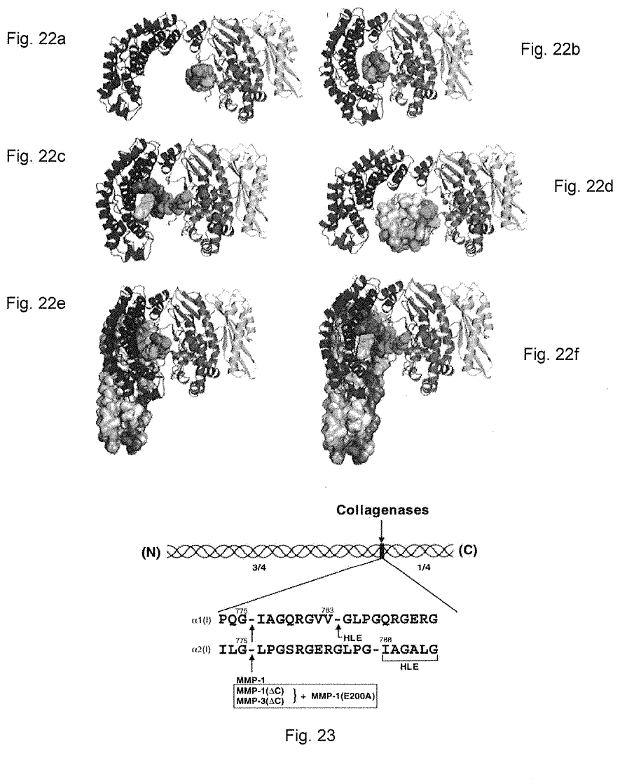

The crystal structure of CoIG and the changes in its conformation during active and inactive states is depicted in FIGS. 22a-22f [149].

In comparison, the ColH enzyme contains two PKD-like domains but only one CBD [150]. ColH also possesses a selectivity loop creating a tube-like compartmentalization of the active site and is unique to ColH where the loop opens when an appropriate substrate is present [150]. Additionally, ColH also contains an aspartic acid ((Asp)421) residue that binds the active site zinc, blocking its accessibility to substrate, coined the “aspartate switch” [150]. The combination of the aspartate switch and the selectivity loop explain the low collagenolytic activity against viable triple helical substrates, which cannot reach the active site due to size, with preferential activity against single chain substrates (denatured collagen) [150].

Though enzymatic activity of both Ccol classes is quite similar to native MMP there are some subtle and important differences. To begin, CoIG and ColH function like native MMPs in that they degrade native collagens at set peptide sequences, but not in the typical three-fourths and one-fourth peptide fragments [21] [149] [155] [78]. The type I and II Ccol enzymes are able to cleave collagen into numerous small peptide fragments at distinct hyperreactive Y-Gly (Glycine) bonds in the repeating Gly-X-Y collagen sequence [21] [78] [155].

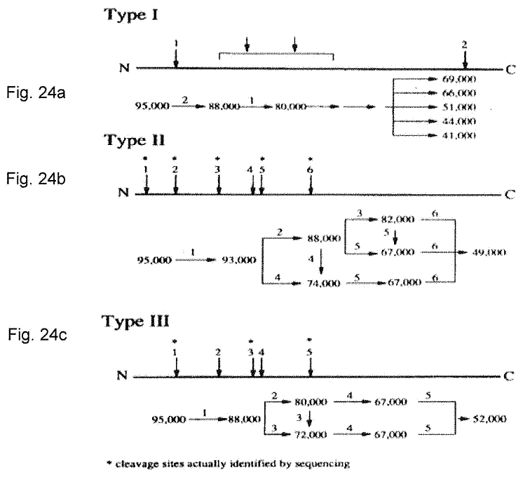

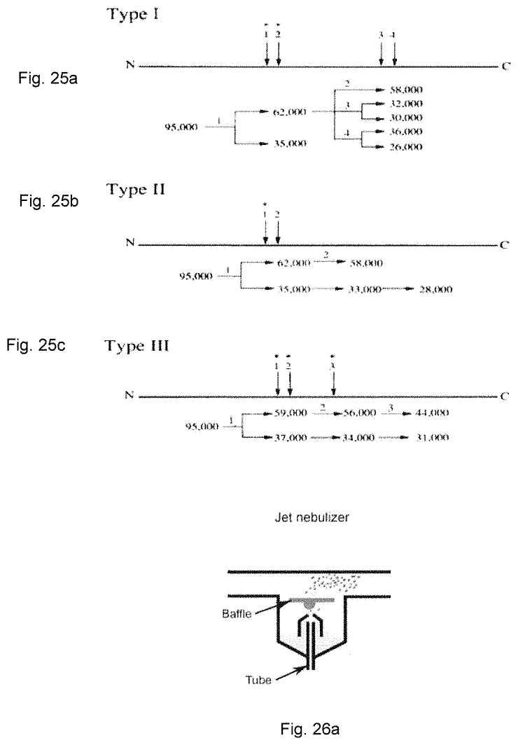

FIGS. 23, 24a-24c, and 25a-25c highlight the differences in collagen cleavage sites between native MMPs and the clostridial derived collagenases (Type I (ColG) and Type II (ColH)) [21, 86].

Despite similar site recognition, ColH preferentially digests collagen at the center of collagen strands versus CoIG's preferential cleavage at the ends of collagen strands [152] [21]. Additionally, CoIG conformational changes allows for more efficient substrate distortion as compared to the MMPs, further enhancing their collagenolytic ability [149]. Like native MMPs Ccol uses hydrolytic entropy to power enzymatic degradation of collagen, which is possible due to collagens well hydrated structure, and is accomplished in a fashion that is independent of triple-helicase and peptidase activity [149, 156]. The combined effect of broad cleavage site's and cooperative enzymatic behavior allow ColH and CoIG to work in a synergistic fashion that is far more expeditious than native MMP activity [157] [158].

Another critical contrast from native MMPs, is that Ccol is relatively incapable of digesting or harming native viable human collagen[152] [21] [2] [20]. French et al, determined that though both CoIG and ColH display specificity at set hyperreactive cleavage sites on collagen, the clostridial enzymes also identify collagen structure that differs locally from that of the remaining collagen chain [152] [21]. Thus viable collagen is unharmed due to its conformational state allowing the enzyme to only preferentially degrade damaged tissue [21] [152]. In-vivo animal study's corroborate these findings, with absolutely zero degradation of type IV collagen bound to intact basement membrane, nor any degradation of laminin, both of which are key component of structural ECM integrity [20]. Additionally, Ccol poses no threat to endothelial cells with no demonstratable hemorrhagic reactions seen in this animal study [20]. This vascular sparing effect is also explained by its inability to digest fibrin, thus limiting clot breakdown, ensuring hemostasis when applied to injured tissue in the earlier stages of healing 20] [159]. In contrast native MMPs, like MMP-1 for example, are only able to degrade native collagen structures and therefore not only cause initial inflammatory insults but also limits their ability to digest fibrotic denatured collagen scaffolds [160]. Related to its therapeutic benefit, Ccol liberated ECM protein fragments increase endothelial and fibroblast proliferation, resulting in improved granulation tissue formation, similar to the effects induced by native MMP's [23]. The enzyme also enhances angiogenic remodeling in vitro by 50-100% when applied to dermal wounds, a factor critical in limiting fibrotic conversion of nonviable tissue during wound healing [23]. These unique ECM derived peptides are created due to Ccol's unique collagen cleavage sites and include thrombospondin (TSN) peptides 1, 2, and 6, in addition to the alpha-3 chain of type VI collagen, TGF-Beta induced protein, tenascin-C as well as multimerin-1 (MMRN-1) [23]. These ECM derived peptides also significantly increased epithelialization by 60-100% [23]. Lastly, Ccol's collagen binding and degradation is not limited in scope as it is capable of degrading and recognizing all types of collagen in the human body in both in-vitro and in-vivo settings, including the lung a stark contrast to native MMP's [24].

### Current Medical Uses of Clostridial Collagenases

The pharmaceutical industry has capitalized on Ccol's preferential digestion of non-viable tissue, with applications to various medical conditions. To begin, dermal supplementation of Ccol works at the cellular level, with 2-fold increase in keratinocyte proliferation and postinjury migration observed on in-vitro skin wounds [3]. In relation, Ccol also improves healing, limits excessive fibrosis and curtails inflammation when applied to epithelial burn wounds for instance [119]. Its use early in burn wounds also resulted in greater cellular migration, reduced apoptosis and subsequent conversion to necrosis when applied early in burn injuries [119]. This same study also found that Ccol expedited early epidermal separation of necrotic tissue with a clearly defined intact basement membrane, improving inflammatory markers and leading to faster wound resolution [119]. Additionally, this study demonstrated greater blood vessel preservation a factor that is necessary to provide necessary oxygen, nutrients for wound healing and limit propagation of injury [119]. The enzyme has profound effects on inflammation enhancing anti-inflammatory cytokine release and reducing the levels of proinflammatory factors [161]. The use of Ccol on diabetic wounds has also shown to have promising results when compared to traditional moistened saline gauze. Use of Ccol on this patient population demonstrated improved wound assessment scores at 4 weeks of treatment (Ccol, −2.5, p=0.007; vs Saline gauze, −3.4, p=0.006), a statistically significant decrease in mean wound area after completion of treatment (p=0.0164) and at subsequent follow-up (p=0.012) [162]. Its use on dermal wounds has also translated to a reduction in healthcare associated costs with Ccol treated stage IV pressure ulcers incurring lower costs (Ccol vs Non-Ccol: $11,151 vs $17,596) and greater ulcer free weeks (Non-Ccol vs Ccol: 33.9 weeks vs 16.8 weeks) [163]. Its use on animal model hypertrophic scars showed reduced new total scar area (Ccol vs Non-CCol: 531±32 vs. 617±51; p=0.03), as well as a lower scar elevation index (Ccol vs non Ccol: 2.08±0.15 vs. 2.45±0.20; p=0.015) [164]. It has also been applied successfully in treating Dupuytren's contracture as well as Peronei's disease, both previously mentioned pathologies of ECM remodeling[165] [166] [102]. Injection of Ccol into Dupuytren's contractures resulted in significant down-regulation of ECM components and inflammatory cytokines in a dose-dependent manner [102]. These changes correlated with reduced expression of type I and III collagens, fibronectin, alpha smooth muscle actin (a-SMA), transforming growth factor beta one (TGF-b1) and matrix metalloproteinase type-9 (MMP-9) levels (p<0.05) [102]. Likewise Ccol has demonstrated similar efficacy in the treatment of pilonidal wounds and significantly improves cellulite appearance and is generally well tolerated with minimal side effects [167] [168] [103].

### Safety Profile of Clostridial Collagenase on Human Subjects

In addition to its therapeutic effects Ccol administration has extensive evidence regarding its safety profile. A meta-analysis in 2019 found that topical application of Ccol on skin wounds was not only effective in debridement of nonviable tissue and accelerating wound healing, but has minimal infection risk and a favorable side effect profile [169]. In relation, no systemic or local reactions attributed to overdosage has been observed in its clinical use [170] [169]. In human studies, after subcutaneous injection no quantifiable systemic Ccol levels were observed at doses up to 3.36 mg [171]. This same study aimed to assess the potential effects of inadvertent intravenous administration of Ccol and found no associated systemic toxicity with dosing up to forty-three times the proposed human dose in animals (mg/kg) being well tolerated [171]. Its use in a rat model did show a reactive transient liver transaminitis that resolved in 14 days with no adverse clinical effects [171]. After a 5 year follow up in patients treated for Dupuytrens contracture only one mild adverse event was reported (skin atrophy leading to decreased ring finger circumference) [172] [166]. This study also found that 93% of those treated with Ccol had circulating antibodies to the enzyme however these antibodies did not correspond to any adverse events [166]. Of the reported adverse effects related to dermally injected Ccol administration the majority are minor localized injection site swelling and pain that is self-limiting, with no serious adverse effects reported [172] [171] [103]. Of note, there has been one isolated hypersensitivity reaction reported in a patient treated for more than a year with the topical form of Ccol however this was in combination with prolonged cortisone administration was well [170]. Lastly, the presence of proteoglycans of which are commonly intimately related to collagen, stabilizes viable collagen and limits MMP and Ccol enzymatic access to collagen cleavage sites, further bolstering its safety profile [173] [174]. This data affirms the tolerability and safety of Ccol with a virtual lack of observed systemic toxicity.

### Feasibility of Nebulized Clostridial Collagenase

The use of nebulized therapy for treating pulmonary disease has expanded greatly since its inception. Targeted pulmonary delivery of medications has innumerous benefits including minimal adverse systemic effects, higher bioavailability, rapid onset of action and lower dosage requirements [175]. Its use in clinical practice has proven to efficacious in the treatment of various conditions ranging from albuterol for asthmatics to cystic fibrosis and COPD, amongst others [175] [176] [177]. However, for a medication to be considered for intrapulmonary delivery it must be effective, tolerable, safe and possess characteristics compatible for nebulized delivery [178]. In relation, the formulation of Ccol in a nebulized form for intrapulmonary delivery is feasible as it possesses these critical traits. As previously discussed, Ccol in crude form, is a lyophilized powdered and is freely soluble in solution [23]. It functions at an optimal pH range of 6.3-8.8 and has demonstrated stability in different diluents including normal saline (0.9% NS) solution with no effect on its enzymatic activity [179] [180] [22] [181] [182]. In relation, inclusion of 2% lidocaine in the diluent and/or reconstituted fluid has no effect on enzymatic activity and can limit possible bronchial hyperresponsiveness during administration [180].

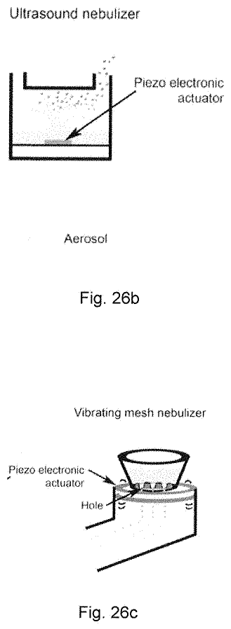

To reach the alveolar interphase lower airway nebulized particles typically need to be in a size range of approximately ˜0.5-5 μm (microns), at a sufficient dose that is not affected by its nebulized delivery method and remain in a non-denatured form to maintain adequate enzymatic ability [175]. This specific particle size requirement is referred to as mass median aerodynamic diameter (MMAD) (the diameter at which 50% of the particles by mass are larger and 50% are smaller) of between 1 and 5 μm of which is required for lower airway deposition [183]. Studies have found that the reconstituted and diluted Ccol is centrifugible to a size of 0.2 to 0.8 μm (microns) with intact activity and stability, making it compatible for nebulized lung delivery [179]. Currently there are various methods available to deliver nebulized medications including metered-dose-inhalers (MDI), soft mist Inhalers (SMI), dry powder inhalers (DPI), surface acoustic wave nebulizers (SAWN), jet nebulizers (JN), ultrasonic nebulizers and vibratory mesh nebulizers (VMN) each with inherent strengths and weaknesses [175]. FIGS. 26a-26c depict the different options available for nebulization drug delivery [184].

However of these options VMNs allow for efficient protein delivery at a size capable of reaching the alveolar level and without heating or risk of denaturing the delivered agent [185] [175]. VMN's utilize a plate mesh with numerous apertures that allows for delivery of medication compounded solutions with high efficiency [184] [186]. VMN particle creation is associated with reduced aerosol loss in ventilator systems, delivers a greater inhaled mass, does not dilute the aerosolized medication during delivery nor require specific air flow, pressure or volume changes for delivery [187] [185] [175] [188]. VMN use reduces the risk of medication loss with less than 10% residual volumes reported, optimizing medication delivery [189] [175]. Additionally, for delivery the medication solution only needs to pass through the device once, reducing shearing of the medication and associated risk of damaging the drug, thus making them ideal for bioactive medication delivery [175]. This nebulization system is capable of delivering stable non-denatured biologically active peptides and has previously demonstrated the ability to maintain 90-100% of inherent enzymatic activity when delivering DNase [190] and alpha 1-antitrypsin [184]. Thus numerous studies have designated VMNs as a dependable and optimal delivery system for deep lung penetrance, being commonly used in clinical trials as well as in every day clinical settings [191] [192] [175].

In relation to pulmonary pathology like ARDS and IPF for instance, the application of supplemental Ccol is largely understudied and can possibly serve as a therapeutic option. In a non-ARDS animal model for instance its application on lung tissue produced no effect on long elasticity or compliance, nor did it alter pulmonary histology [193]. In contrast when applied to lung tissue at higher mechanical stressed states, increased strain in the presence of Ccol resulted in less distortion of the alveoli [194]. This coincided with an irreversible decline in lung stiffness on microscopic analysis, suggesting that mechanical injury as evident by barotrauma or volutrauma during ventilation leads to ECM damage allowing Ccol to influence ECM collagen architecture when injury is present[194]. These findings again illustrate Ccol's inability to digest native undamaged collagen tissue as previously demonstrated [21] [152] [20].

Despite improvements in ARDS management, this disease continues to have minimal treatment options and excessive associated mortality and morbidity. The provided evidence suggests that ECM composition in the form of excessive collagen deposition and local MMP deficiency is not only intimately related to ARDS progression but influences mortality and distortion of pulmonary architecture as well as function. Given the clinical utility observed in utilizing Ccol in treating other ECM associated pathological conditions, its low side effect profile, the inability to damage healthy tissue and the lack of effective treatment options for ARDS, application of Ccol to ARDS patients would provide a novel therapeutic agent. The present invention describes the use of CCol in a novel nebulized formulation.

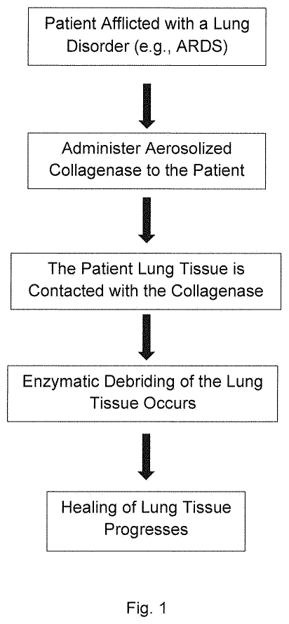

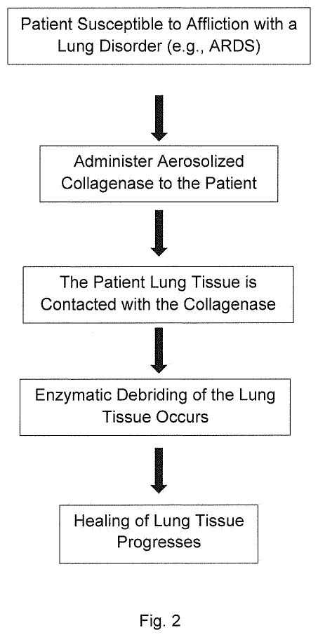

The presently disclosed subject matter includes methods of treating a patient having a lung disorder (e.g., ARDS) that include the administration of a therapeutically effective amount of one or more collagenases by inhalation, as illustrated in the schematic of FIG. 1. The aerosolized formulation is delivered directly to the peripheral airways and lungs of the patient. As such, the disclosed method significantly increases delivery of the collagenase to the lung tissue, thereby improving efficacy of treatment. The aerosolized collagenase acts as an enzymatic debrider, removing dead tissue from the lungs to allow lung tissue healing to progress. The disclosed methods can also be used to prevent onset or progression of a lung disorder (e.g., ARDS), as illustrated in FIG. 2.

At steps 5, 6, and 10, a patient afflicted with a lung disorder is administered aerosolized collagenase. The term “patient” broadly refers to any subject in need of treatment. Thus, the patient can be a human with ARDS or a human susceptible to developing ARDS. However, the patient is not limited and the presently disclosed subject matter can be used with veterinary purposes for the treatment of dogs, cats, goats, horses, ponies, donkeys, rabbits, and the like.

As shown in FIGS. 1 and 2, the patient is afflicted with or susceptible to a lung disorder. The term “lung disorder” refers to any condition characterized by weakness or damage to lung tissue. For example, typical lung disorders can include (but are not limited to) ARDS, COPD, lung cancer, asthma, cystic fibrosis, emphysema, bronchitis, bronchiectasis, interstitial lung disease, interstitial fibrosis, bacterial pneumonia, viral pneumonia, fungal pneumonia, parasitic pneumonia, mycobacteria-caused pneumonia, occupational lung diseases (e.g., those caused by agents such as coal, silica, asbestos, and isocyanates), systemic lupus erythematosus, rheumatoid arthritis, scleroderma, dermatomyositis, mixed connective tissue disorder; vasculitis associated lung disease (such as Wegener granulomatosis and Good-pasture's Syndrome), sarcoid, and/or Acute Lung Injury.

At step 10, a therapeutically effective amount of an aerosolized collagenase is administered to the patient. The term “administered” refers to any form of delivery where the aerosolized collagenase is delivered to the lungs, such as by nasal or oral inhalation.

As used herein, the term “collagenase” refers to one or more proteolytic enzymes capable of enzymatically cleaving collagen. Collagen is the main structural protein of the various connective tissues in animals (e.g., lung tissue). The term “collagenase” does not imply any specific limitations on the type or origin of the collagenase. Thus, a suitable collagenase can be recombinant or from its natural source. Non-limiting examples of a mammalian collagenase suitable for use with the presently disclosed methods include (but are not limited to) mammalian MMPs, such as MMP1, MMP2, MMP3, MMP7, MMP8, MMP9, MMP10, MMP11, MMP12, MMP13, MMP14, MMP15, MMP16, MMP17, and microbial MMPs.

Aerosolized collagenase refers to collagenase in the form of microscopic solid or liquid particles dispersed or suspended in air or gas. Specific information regarding formulations that can be used in connection with aerosolized delivery devices are described within Remington's Pharmaceutical Sciences, A. R. Gennaro editor (latest edition) Mack Publishing Company, incorporated by reference herein.

The aerosolized collagenase includes free flowing collagenase particulates having a size selected to permit penetration into the alveoli of the lungs, generally being less than 10 μm in diameter. Thus, the size of the aerosolized collagenase can be at least about (or no more than about) 0.1-10 μm in diameter (e.g., about 0.1, 0.5, 1, 2, 3, 4, 5, 6, 7, 8, 9, or 10 μm). However, the presently disclosed subject matter is not limited, and the size of the aerosolized collagenase particulates can be outside the range given herein. In any case, the aerosolized collagenase employed should be of a size that is adapted to penetrate to the patient lung.

The collagenase disclosed herein can be administered at a therapeutically effective dosage (e.g., a dosage sufficient to provide treatment for ARDS or a lung disorder as previously described). While optimum human dosage levels have yet to be determined for aerosol delivery, generally a daily aerosol dose of collagenase can be from about 0.1 to 10 mg/kg of body weight. Thus, the dosage can include at least about (or no more than about) 0.1, 0.5, 1, 2, 3, 4, 5, 6, 7, 8, 9, or 10 mg/kg of body weight. For example, for administration to a 70 kg person, the dosage range would be about 7.0 to 700.0 mg per day. The amount of collagenase administered will be dependent on the patient and disease state being treated, the severity of the affliction, the manner and schedule of administration, and the judgment of the prescribing physician.

In some embodiments, the disclosed formulation can comprise about 0.01-90 weight percent active agent (e.g., one or more collagenases). Thus, the formulation can comprise about 0.001, 0.01, 0.1, 1, 2, 3, 4, 5, 10, 15, 20, 25, 30, 35, 40, 45, 50, 55, 60, 65, 70, 75, 80, 85, or 90 weight percent collagenase, based on the total weight of the formulation.

Pharmaceutically acceptable compositions include solid, semi-solid, liquid and aerosol dosage forms, such as, e.g., powders, liquids, suspensions, complexations, liposomes, particulates, or the like. In some embodiments, the disclosed aerosol collagenase compositions are provided in unit dosage forms suitable for single administration of a precise dose. The unit dosage form can also be assembled and packaged together to provide a patient with a weekly or monthly supply and can also incorporate other compounds such as saline, taste masking agents, pharmaceutical excipients, and other active ingredients or carriers.

The collagenase can be administered alone or with a carrier. The term “carrier” refers to a compound or material used to facilitate administration of the collagenase (e.g., to increase solubility). Suitable carriers include (but are not limited to) sterile water, saline, buffers, non-ionic surfactants, or combinations thereof. In addition, various adjuvants such as are commonly used in the art may be included. These and other such compounds are described in the literature, e.g., in the Merck Index, Merck & Company, Rahway, N.J., incorporated by reference herein. Considerations for the inclusion of various components in pharmaceutical compositions are described, e.g., in Gilman et al. (Eds.) (1990); Goodman and Gilman's: The Pharmacological Basis of Therapeutics, 8th Ed, Pergamon Press., incorporated by reference herein.

The disclosed aerosol formulation may be administered in an aqueous solution that is pharmaceutically acceptable for administration to the respiratory system. Thus, the compound can be administered through inhalation in a form such as liquid particles and/or solid particles. Various suitable devices that can be used to administer the aerosolized collagenases to a patient's respiratory tract are known in the art. For example, nebulizers create a fine mist from a solution or suspension, which is then inhaled by the patient. The devices described in U.S. Pat. No. 5,709,202 to Lloyd, et al., or in U.S. Pat. No. 6,615,824 of Power can be used. An MDI typically includes a pressurized canister having a meter valve, wherein the canister is filled with the solution or suspension and a propellant. The solvent itself may function as the propellant, or the composition may be combined with a propellant, such as freon. The composition is a fine mist when released from the canister due to the release in pressure. The propellant and solvent may wholly or partially evaporate due to the decrease in pressure.

The formulations of the pharmaceutical compositions described herein may be prepared by any method known or hereafter develop in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

At step 25, the patient is treated with the collagenase. As used herein, the terms “treating” or “treatment” refers to the alleviation, suppression, repression, elimination, prevention, or slowing the appearance of symptoms, clinical signs, or underlying pathology of a lung condition or disorder (e.g., ARDS) on a temporary or permanent basis. For example, symptoms of lung injury and/or inflammation include reduced pulmonary gas exchange, reduced pulmonary shunt fraction, extracellular fibrin deposition, increased vascular permeability, decreased lipoprotein surfactant deposition, tissue remodeling, coagulation, and/or increased alveolar tension. Preventing a condition or disorder involves administering a formulation comprising aerosolized collagenase to a patient prior to onset of the condition. Suppressing a lung condition or disorder involves administering a formulation as disclosed herein to a patient after clinical appearance of the condition or disorder. Prophylactic treatment may reduce the risk of developing the lung condition and/or lessen its severity if the condition later develops. For instance, treatment of an existing ARDS condition may reduce, ameliorate, or altogether eliminate the condition, or prevent it from worsening.

The aerosol is preferably administered orally, nasally, or oro-nasally. Additional modes of administration are possible, as disclosed herein. Thus, the aerosol can be inhaled through the patient's mouth, nose, or both.

The compositions are delivered into the lung with a pharmacokinetic profile that results in the delivery of an effective dose of the collagenase. As generally used herein, an “effective amount” of a collagenase as used herein is an amount capable of treating one or more symptoms of a lung disease, reverse the progression of one or more symptoms of a lung disease, halt the progression of one or more symptoms of a lung disease, prevent the occurrence of one or more symptoms of a lung disease, decrease a manifestation of the disease, or diagnose one or more symptoms of a lung disease in a patient to whom the compound or therapeutic agent is administered, as compared to a matched patient not receiving the aerosolized collagenase.

The therapeutically effective amount can be routinely determined by one of skill in the art, and will vary depending on several factors, such as the patient's height, weight, sex, age, and medical history. For prophylactic treatments, a therapeutically effective amount is that amount effective to prevent a lung disorder (e.g., ARDS) from occurring.

In some embodiments, a dosage of aerosolized collagenase can be administered to a patient as frequently as several times daily. Alternatively, a dosage can be administered less frequently, such as once a day, once a week, once every two weeks, once a month, once every several months, or even once a year or less. The frequency of the dose will be readily apparent to the skilled artisan and will depend upon any number of factors, such as (but not limited to) the type and severity of the disease or disorder being treated, the sex, health, and age of the patient. Thus, treatment can continue for any desired period of time, such as until the symptoms of the lung disorder are eliminated or improved. As it will be recognized in the art, the duration of treatment can and will vary depending on the progress of treatment.

It is understood that as further studies are conducted, information will emerge regarding appropriate dosage levels for treatment of various conditions in various patients, and one of ordinary skill in the art, considering the therapeutic context, age, and general health of the recipient, will be able to determine proper dosing.

Toxicity and therapeutic efficacy of collagenase aerosols can be determined by standard pharmaceutical procedures in cell cultures or experimental animals (e.g., for determining the LD50 (the dose lethal to 50% of the population) and the ED50 (the dose therapeutically effective in 50% of the population)). The dose ratio between toxic and therapeutic effects is the therapeutic index and it can be expressed as the ratio LD50/ED50. While compounds that exhibit toxic side effects can be used, care should be taken to design a delivery system that targets the collagenase to the site of affected tissue to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. It should be appreciated that the dosage can vary within this range depending upon the dosage form employed and the route of administration utilized. The therapeutically effective dose can be estimated initially from cell culture assays. In some embodiments, a dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses more accurately in humans.

In some embodiments, the disclosed aerosolized formulations can be administered in conjunction with one or more treatment regimens for ARDS, such as the use of a ventilator, delivery of antibiotics, and the like.

Advantageously, aerosolized delivery of the collagenase to the lungs of the patient promotes recovery. Specifically, at step 20, enzymatic debriding of the lung tissue occurs. Without being bound by any particular theory, the disclosed formulation when administered to a patient in an inhaled aerosolized form functions to dissolve lung scar tissue that adversely affects oxygen exchange, whether it be at the alveolar capillary interface or the lung parenchyma itself with minimal local damage to the alveoli. As a result, patient outcome is improved and the mortality rate in patients with ARDS is decreased, as indicated in step 25.

The disclosed methods can be practiced to alleviate and/or treat ARDS in an individual diagnosed with ARDS. The methods can also be used as a prophylactic treatment in an individual at risk for developing ARDS. The presently disclosed subject matter further contemplates alleviation and/or treatment of other respiratory conditions by administering a therapeutically effective amount of aerosolized collagenase to the patient. Other respiratory conditions include (but are not limited to) idiopathic interstitial pulmonary fibrosis, chronic obstructive pulmonary disease (COPD), and/or asthma.

In some embodiments, the presently disclosed subject matter can include a kit for treating lung disorders, such as ARDS. For example, the kit can include a therapeutically effective amount of an aerosol form of collagenase, and instructions for use. The aerosol can further include a pharmaceutically acceptable carrier, such as water or saline. In some embodiments, the kit can include a nebulizer system (e.g., jet aerosol, ultrasonic nebulizer, or dry powder inhalation system).

The presently disclosed system and methods provide advantages over prior art treatment methods. For example, the disclosed method can be used to treat a patient with little or no discomfort or adverse side effects.

Use of the aerosolized collagenase compositions function to break down lung scar tissue, while avoiding or minimally damaging normal surrounding healthy tissue.

Further, minimal training or knowledge is needed to administer the disclosed method.

Exemplary embodiments of the methods and components of the presently disclosed subject matter have been described herein. As noted elsewhere, these embodiments have been described for illustrative purposes only, and are not limiting. Other embodiments are possible and are covered by the presently disclosed subject matter. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter. All cited references are hereby incorporated by reference in their entireties.

### Prophetic Example 1

### Formulation of a Clostridial Collagenase (Ccol) Solution Suitable for Nebulization to the Level of the Alveolar Lung Interphase

The solution will be formulated at concentrations ranging from 0.1 to 10 mg/ml or greater if necessary as exemplified previously[179] [180] [195]. This will include crude applications of the clostridial enzyme as well as, or purified clostridial derived collagenase. The lyophilized powder will be reconstituted and diluted with various solutions as previously described, (i.e., QWO proprietary diluent (0.6% sodium chloride (NaCl) and 0.03% calcium chloride dihydrate (CaCl₂)) and/or Xiaflex® diluent 3 mL of 0.3 mg/mL calcium chloride dihydrate in 0.9% sodium chloride), or in various concentrations of normal saline (NS) solution (0.9% NaCl), which includes an osmolarity of 308 mOsmol/L, 154 mEq/L sodium and 154 mEq/L chloride, ranging from (0.1-0.9%) NS [196] [172] [180] [197].

As a quality control measure, Ccol enzymatic activity will be assessed after its reconstitution in various excipients prior to intrapulmonary nebulized delivery. This is typically conducted combining stock enzyme solution and dissolving 0.05-0.1 mg/ml collagenase in 50 mM TES buffer, pH 7.4 (37° C.), containing 0.36 mM calcium chloride; yielding a final concentrations in the reaction mixture of 50 mM TES, 0.36 mM calcium chloride, 25 mg collagen (Product No. C 9879), and 0.005-0.01 mg collagenase [198] [199] [200] [201] [179]. Alternatively, enzymatic activity may be assessed by radiolabeling of collagen substrates as previously described [202] [199]. Lowering the enzyme concentration by administering bovine serum albumin (BSA) or serum (0.5% and 5-10%) respectively may be used to titrate concentration if necessary during enzymatic analysis, amongst others [198] [199] [200] [201] [179] [202]. As the crude form has additional enzymes in the compound their activity will be assessed as described by Mandl et al [203]. These include neutral protease (caseinase) activity will be assessed as follows: ≤350 units/mg solid (Unit Definition: One Neutral Protease Unit hydrolyzes casein to produce color equivalent to 1.0 μmole tyrosine per 5 hours at pH 7.5 at 37° C.). Clostripain activity: ≤4 units/mg solid, Unit Definition: One Clostripain Unit hydrolyzes, 1.0 μmole of BAEE per minute at pH 7.6 at 25° C. in the presence of DTT. Likewise purified collagenase enzymes type I and type II may be alternatively used.

The purity of the compound in solution will be assessed by densitometry and integration of bands observed following reduced SDS-PAGE as previously described [180]. This will be accomplished utilizing NuPage™ 4-12% Sodium Dodecyl Sulfate Bis-Tris Polyacrylamide Gel (Invitrogen #NP0322-BOX) with Coomassie Blue staining [180]. Changes reported for each condition are relative to its respective baseline sample [180]. High performance liquid chromatography at 280 nm will be used to assess aggregation and enzyme stability for both Ccol enzymatic components (Collagenase type I and type II) [180]. This may be determined using size-exclusion analysis amongst others [180]. Aggregation will be determined by peak area integration for each sample relative to reconstitution with each diluent and or excipient [180].

Ccol enzymatic activity after dilution with the various solutions and diluents will be assessed as previously conducted [180] [204]. Various synthetic peptides, which are similar in structure to collagen will be used for this assessment as included in the following references [205] [206] [182] [207] [208] [203, 209] [210] [211].

Enzyme activity will be determined relative to reference standard activity in Mandl units which have equivalency as what is reported with Sigma collagen digestion units, with a conversion factor for Mandl units/Wuensch units to Sigma units (approximately 1000-2000 to 1) [203] [208] [179] [180]. The definition of one collagen digestion unit (CDU) liberates peptides from collagen equivalent in ninhydrin color to 1.0 μmole of L-leucine in 5 hours at pH 7.4 at 37° C. in the presence of calcium ions [203] [208] [179] [180]. Activators/Cofactors include 0.1 mole calcium ions (Ca2+) per mole of enzyme as calcium ions also facilitate binding and stabilize the enzyme [212] [195]. Zinc ions (Zn2+) are required for activity, but are tightly bound to the collagenase during purification, additional Zn2+ may be required if a chelator has been included in the admixture [213]. Additional assays for enzymatic activity via proven calculations may alternatively be used as previously described [214] [215] [216].

The figures included in the cited reference provides a comparative example of the methods and results used to assess clostridial collagenase enzymatic activity, its assessment of purity and evaluation of aggregation in various solutions [180].

SDS Page Purity Assay: sample purity will be assessed by densitometry and integration of bands observed following reduced SDS-PAGE. SDS-PAGE conditions utiltizing a NuPage 4-12$ SDS Bis-Tris polyacrylamide gel (Invitrogen NP0322-BOX) with Coomassie Blue staining. Changes reported for each condition are relative to its respective baseline sample (to).

Size-exclusion chromatography for determining aggregation and stability: Samle AUX-I and AUX-II content and any protein aggregation can be determined using size exclusion HPLC at 280 nm (Agilent 1100 System with Superdex 200 10/300 GL column, Cat. No. 17-5175-01). Protein aggregation can be determined by peak area integration for each sample relative to reconstitution with proprietary diluent plus a saline diluent in glass at to.

Enzyme activity assays: collagenase (AUX-I) enzyme activity was evaluated using serial dilution of a commercially available peptide substrate (Glycine-Proline-Alanine) and Gelatinase (AUX-II) enzyme activity can be evaluated using serial dilutions of a commercially available soluble rat collagen as substrate as previously described. Enzyme activity will be determined relative to a reference standard and any changes in activity will be reported for each condition relative to reconstitution with proprietary diluent plus a saline diluent in glass at t₀. [180]

Inhibitors of Clostridial Collagenase that may be administered during this process are listed below, of which have also been previously described and include [217] [218] [219]: Ethylene glycol-bis(2-aminoethylether)-N,N,N′,N′-tetra-acetic acid (EGTA); Ethylenediaminetetraacetic acid (EDTA). Additionally, the use of EDTA may serve as an antidote in nebulized form, as it has demonstrated utility and safety as an adjunct compound for nebulized medication formulation.

β-Thujaplicin (Hinokitiol), Cysteine, 2-mercaptoethanol, Glutathione (reduced), Thioglycolic acid, and Sodium 8-Hydroxyquinoline-5-sulfonate can be used.

Additional excipients/diluents that may be used during compound formulation are described [52]: amino acids (leucine, glycine, alanine, methionine, tryptophan, tyrosine), small carbohydrates (lactose, mannitol, trehalose, sucrose), polysaccharides (dextran, HA, chitosan), synthetic polymers (PVP K25, PVP K30, EC, PS 20, PS 80, PX 188, solutole, PEG 300, PEG 200, PEG 400, PEG 600, PLGA, NaCMC, starch), surfactants (Brij-35, SorbMO), phospholipids (DPPC), and miscellaneous (FDKP, CD, AB, NaCl, NaCit, NaAlg, glycerol, ethanol) [52].

### Prophetic Example 2

### Determination of the Efficacy, Toxicity and Cytotoxicity Profile of the Formulated Nebulized Clostridial Collagenase Compound Solution in Preclinical Studies

Preclinical Animal Studies: nebulized Ccol delivery will be assessed in preclinical animal models to determine adequate dosage, safety and efficacy [220]. For this analysis and all subsequent animal model analysis the medication will be administered via direct instillation, intratracheal, intranasal or nose-only aerosol inhalation, amongst others [221]. End point efficacy data will be generated after assessment of the methods [221].

A comparative example of various postulated animal models that may be used in relation to human characteristics are provided in Table 4 of [52].

To assess safety of the compound for particle aerosolization both preliminary in-vitro, in-vivo and/or ex-vivo testing may be conducted [52]. Animals will be assigned into respective groups at random including: a control group of which will receive no treatment, a placebo group of which will only receive normal saline, and treatment groups of escalating drug concentration ranges [222]. Efficacy and toxicity for delivery to the pulmonary system will be assessed with the use of these animal models in both an in-vivo and in-vitro fashion [52]. In-vitro cytotoxicity trials will be initially conducted on animals and/or human derived alveolar epithelial cell isolates, with application of the formulated Ccol solution [223]. In-vivo assessment will be performed concurrently or following initial in-vitro cytotoxic assessment. Assessments will include predefined sampling of serum, BAL, histopathological analysis and inter-group comparison as well as observation for any adverse responses at various dosages [222].

Table 3 of [193] and FIG. 7 and Table 2 of [223] serve as comparative examples of this process [223] [193].

FIGS. 1, 2 and Tables 1-2 of [222] serve as comparative examples of a related study method.

### Prophetic Example 3

### Assessing the Optimal Delivery Method, Nebulized Particle Size at Various Dosages, Lung Penetrance, Alveolar Clearance, and Distribution of the Nebulized Clostridial Collagenase Compound in Nebulized Ventilation Simulation Models as Well as Animal Models

To assess the performance of the formulation aerodynamic particle size distribution (APSD) will be evaluated using a twin-stage impinger (TSI) or a multi-stage liquid impinger (MSLI) which can separate particles at different stages and per size [224] [225] [52]. Alternatively, this may be accomplished via use of cascade impactors including: Andersen Cascade Impactor (ACI) or the Next Generation Impactor (NGI) [225] [52]. The use of in-vitro nebulizer simulation models may also be employed to assess nebulized performance for compound delivery[187].

FIG. 2 and Table 2 of [187] depict a comparative example of a previously conducted study on inhaled colistin in an in-vitro simulation model using a cascade impactor.

Particle inhalability, will be calculated based upon previously derived equations [226, 227]. Predicted deposition efficiency may be calculated as a percentage of the mass of unit density spheres entering the respiratory tract or oropharynx [226]. Ccol compound will be nebulized with a goal mass median aerodynamic diameter (MMAD) (the diameter at which 50% of the particles by mass are larger and 50% are smaller) of between 1 and 5 μm of which is required for lower airway deposition[187] [183]. This can be accomplished with the use of currently available standardized vibratory mesh nebulizer (VMN) system of which utilize a plate mesh with numerous apertures that allows for delivery of medication compounded solutions with high efficiency [184] [186]. This nebulization system is capable of delivering stable non-denatured biologically active peptides and has previously demonstrated the ability to maintain 90-100% of inherent enzymatic activity when delivering DNase [190] and alpha 1-antitrypsin [184]. The performance of VMN delivery as well as other nebulization delivery devices/methods of the formulated compound will assess residual drug mass, volumetric median diameter (VMD) as a measure of aerosol droplet size using laser diffraction as previously described[187] [228].

A comparative schematic of a simulated nebulization protocol apparatus is illustrated in FIG. 1 of [187]. A comparative example of Nebulizer Performance measures on inhaled colistin is shown in Table 1 of [187]. Table 2 of [183] serves as a comparative example of previously conducted assessments of fine particle fraction and particle size determination for drug nebulization.

### Animal Model for Nebulized Particle Assessment

Preliminary results on small animal trials and simulated ventilation nebulizer efficacy studies will be extrapolated to additional small animal or larger sized animal study models to assess lung deposition and distribution of the nebulized Ccol compound in-vivo and/or ex-vivo [52]. One commonly used ex-vivo model is the isolated perfused lung (IPL) method typically conducted on rats, which involves isolating lung and pulmonary circulation [224] [225] [229] [52]. The use of this model allows for assessment of pulmonary uptake and metabolism of administered compounds[224] [225] [229] [52]. Lung deposition and dosing will be assessed via the use of Multiple Path Particle Dosimetry model (MPPD) [187, 228]. Lung deposition of inhaled substances depends on the route, rate, depth of breathing (tidal volume), the volume deposited in the upper respiratory tract (URT), as well as the volume of the lungs at functional residual capacity (FRC) [230]. Deposition may involve possible cross-species conversions based on previously derived models for MPPD involving breathing frequency formulations [230].

Tables 4, 8, and 9 of [230] serve as comparative examples of previously utilized cross-species breathing frequency and FRC conversions. Similar studies and experiments will be performed.

FIGS. 2 and 3 of [230] serve as comparative examples related to lung particle distribution and deposition analysis on humans and various species (mice, Sprague-Dawley rats, and humans). Similar tests will be performed.

Dose predictions, deposition measurements, port uniformity for air flow rates, in cohesion with lung deposition analysis (possibly via fluorescence imaging) as well as predictive measurements of dosing and deposition will be derived at various concentrations and formulations as previously described [226]. Alternatively in-vivo imaging techniques including gamma scintigraphy, single photon emission computed tomography (SPECT) and positron emission tomography (PET) may be used to assess lung particle distribution [52]. Alveolar clearance rates of the compound will be assessed via the MPPD model, where initial mass burden at the end of exposure=mA0, and γ, α, β, and τ are all constants estimated from serial data measurements.

The MPPD model for alveolar clearance is described by the following equation [230]:

λ=γ+α exp(β(m_{A0}^{τ})

In addition to the above methods, the aerodynamic diameter of nebulized particles (diameter of a unit-density sphere that has the same settling velocity as the measured particle) may also be assessed via particle size delivery [52]. This is a proven model to calculate deposition based on particle morphology, the properties of the particle, the concentration and duration to exposure, as well as its clearance.

The relationship between aerodynamic diameter (da), geometric diameter (dg), and particle density (Pp), is described by said equation[52]:

$${d}_{a} = {d}_{g} \sqrt{\frac{{ρ}_{p}}{{ρ}^{*}}}$$

The equation can also incorporate density of the particle (p*) for spherical calibration particle and for non-spherical particles [53] [230]. Particle deposition pattern and particle size distribution based on previously mentioned surface areas and may include other ventilatory/nebulization systems as previously described [228]. Drug delivery measures and aerosol dose efficiency during simulated invasive mechanical ventilation at various settings and its influence on residual drug measurements, ventilator circuit pressure and flow measurements will be assessed [228]. This may include use of enhanced condensational growth (ECG) and excipient enhanced growth (EEG) to increase deposition of particles in a targeted fashion [52]

### Dosing Equivalency Conversion from Animal to Humans

In coinciding with animal cytotoxicity and lung deposition trials the nebulized Ccol will be assessed for its efficacy, side-effect measurements and toxicity to obtain a final therapeutic index (TI) for the inhaled compound [221]. These effects will be measured in similar animals after inhaled administration at escalating dose, to a level at least 10 times greater than the efficacious dose deposited in the lungs however this may vary based on the response [221]. After determining an adequate TI of greater than 10 or of an adequate safety margin, inhaled toxicology studies will be performed to set the maximum dose allowed in human clinical trials [221].

FIGS. 1 and 3 of [221] serve as comparative examples of known efficacy and dosing study models previously used as well as graphical representations for determining therapeutic and toxicity dose limits, for subsequent TI (Therapeutic Index) derivation.

To convert equivalent dosing in animal studies to humans the dose adaptation factor (DAF) will be calculated [53]. This is value is derived using the ratio of minute ventilation (VE; animal/human) multiplied by the ratio of deposition fraction (DF; animal/human) and by the normalization factor (NF; area human lung/area animal lung). To perform this computation NF values of approximately 62.7 m² for humans and 0.409 m² for rats will be used with possible variations in these values by ranges of 1.8 times for animals and 2.5 for humans [53] [231]. Surface area values used for computation have had previously described ranges of 57.22-102 m² for humans alveolar surface and 0.297-0.40 m² for rats [53] [232]. Alternatively, this can be determined via allometric dose scaling [221] [233]. Allometric dose scaling allows for interspecies correlation and is the standard way to approximate equivalent interspecies doses [221]. This equation serves as a useful adjunct for preliminary calculation of the effective human dose [221]. This allometric interspecies scaling equation is as follows: Xh=Xa (Ma/Mh)(1−b), where Xh is the human drug dose (μg) normalized to body mass (μg/kg), Mh is the human body mass (kg), Xa is the animal drug dose per unit body mass (μg/kg), Ma is the animal body mass (kg), b is the allometric exponent [221]. The average of the allometric exponents obtained in mouse, rat and humans supports the current method of scaling using a fixed allometric exponent of 0.67 [221]. The various formulated compounds will then undergo randomized control blinded trials via single dose escalation method as previously described on human subjects [234] [235].

Table 1 of [221] serves as a comparative example of previous uses of the allometric equation used for human dose calculation.

### Prophetic Example 4

### Assessing the Clinical Effect of Nebulized Ccol on ARDS Related Pathology in Animal Models

An ARDS animal model will be applied to assess the clinical effects of the formulated medication in comparison to controls [236] [237]. Table 4 of [238] highlights the different animal models commonly utilized for ARDS and serves as a comparative example.

The animal subjects will be intubated and ventilated using a volume cycled ventilator [123]. The subjects will be placed into different ventilator strategies at random including the following:

1) Standard ventilatory strategy consisting of 3 cm H₂O positive end expiratory pressure (PEEP), a tidal volume (Vt) of 8-10 cm\hat{ }3/kg, and a respiratory rate of 40 breaths per minute (bpm) with room air [123].
2) Lung protective ventilation with Vt of 4-6 cm\hat{ }3/kg, with low PEEP=5 cm H2O [123].
3) Lung protective ventilation with Vt of 4-6 cm\hat{ }3/kg and high PEEP=10 cm H2O [123].

### Assessment of Nebulized Ccol on ARDS Histological/Immunohistochemistry Characteristics

Animal studies will involve the described method. At the time of necropsy half of the specimens will be preserved for subsequent molecular analysis and the other half will be fixed in 10% neutral-buffered formalin or 4% paraformaldehyde (PFA) for 24-48 hours prior to paraffin sectioning [239] [240]. To obtain optimal samples for qualitative and histological analysis the lungs will be inflated, with the subject in the supine position, via a ventral tracheotomy with neutral buffered 10% formalin or 4% paraformaldehyde at room temperature under a constant pressure of 15 to 25 cm H₂O for gross and histological staining [239] [240]. The trachea will then be tied off midway between the larynx and carina to preserve inflated architecture [239]. En-bloc removal of the heart and lungs will be performed and the specimen will be submerged in fresh formalin for approximately 24-48 hrs [239]. The specimen will be placed on the histological cassette with the ventral lobar surface face down in the cassette. Sectioning will be performed longitudinally and parallel to main lobar axis [239]. All embedded tissue containing paraffin will be sectioned in 7 mm portions and counterstained with hematoxylin and eosin (H&E) or massons trichrome after deparaffinization and rehydration for slide analysis [239] [240]. Three nonconsecutive H&E slides from each subject at the level of the alveoli will be obtained at the pre-set time periods [239] [240]. Additionally, histological lung injury scores will be assessed at preset time points and compared between both groups [239].

Table 3 of [239] highlights the components of the histological lung injury score.

FIG. 1 of [239] serves as a comparative example of animal model necropsy utilized in rat models that can be performed.

### Assessment of Nebulized Ccol Activity on Lung Collagen Content

Activity at various time points during preclinical trials the effect of nebulized ccol will be compared to controls for its effect on collagen content. This can be accomplished via various previously described methods, including the relative expression of type 3 and type 1 collagen and its subsequent degradation products in both BAL and serum, assessment of collagenase activity, as well as H & E slide analysis of lung homogenates as previously described [126, 194] [8, 17] [239] [240] [8].

FIGS. 3 and 5 of [8] provide a comparative example that can be performed.

### Assessment of Nebulized Ccol on Pulmonary Biomechanical Properties

Cytoskeleton (CSK) stiffness will be assessed by magnetocytometry (MTC) as described previously [17, 241]. As an internal control Cytochalasin D (cyto D) will be used as it induces a marked decrease in CSK stiffness by disrupting filamentous actin [17] [242]. Following the 24-hour incubation with or without CCol, cell stiffness will be measured by MTC, cyto D will then be added for 20 minutes and cell stiffness will then again be measured [17]. The difference of cell stiffness before and after cyto D will be calculated and reported as change in dynes/cm2 [17]. Additionally, static compliance graphs, pressure-volume curves will be recorded during the before and after nebulized Ccol as well as the control at interval set time points to further assess influence on ventilatory mechanics as previously described[126, 194, 243] [123].

### Assessment of Nebulized Ccol on Pulmonary Gas Exchange and Physiologic Dysfunction

After administration of nebulized Ccol at various time periods various physiologic parameters will be evaluated[239, 243] [237]. These will include relative levels of hypoxemia, changes in alveolar-arterial oxygen gradient, changes in PaO2/FIO2 ratios, minute ventilation, as well as respiratory rate amongst others[243] [239] [237] [126].

Table 1 of [126] serves as a comparative example that can be performed.

### Assessment of Nebulized Ccol on Inflammatory and Anti-Inflammatory Cytokine Response

Cytokine ELISAs and lung lavage total protein concentration will be assessed at predetermined time points after administration of nebulized Ccol and control [8, 239, 243] [123] [237]. These will include serum analysis and BAL levels of TNF-α, IL-1b, IL-6, IL-10, IFN-γ, MIP-2(Macrophage inflammatory protein) will be carried out using commercially available ELISA kits and or mRNA expression via quantitative PCR or additional expression assay [8, 239, 243] [244] [123] [237]. Gene expression will then be normalized to its control sample at each time point. The subjects will also have BAL performed, with the attained effluents subsequently pooled and centrifuged for analysis as well [239, 243]. Amount of protein content as well as different cellular expression will be assessed as previously described[239, 243] [123].

Comparative examples as set out in FIG. 2 and Table 2 of [8] can be performed.

### Prophetic Example 5

### Assess the Clinical Effect of Nebulized Ccol on ARDS Related Pathology in Humans

### Human Methods

The nebulized Ccol compound will be applied to human subjects via the use of single dose escalation trials [235]. This will be conducted in a double blinded randomized control trial. Patients will be randomly assigned to receive either nebulized placebo (saline), control (receiving no medication) or nebulized Ccol. The timing of administering the medication will also be assessed and grouped at random and may include the following distinctions: 1) within 48 hours of ARDS diagnosis, 2) after 48 hours and within 7 days after ARDS diagnosis, 3) After 7 days from diagnosis of ARDS, amongst others. The medication and placebo will be labeled without designation of contents. The trial will use concealed allocation, with randomization done in blinded fashion as well. Each medication vial will have a unique number code and will be labeled before shipment to the clinical site. This code registry will be maintained at a remote central location to maintain integrity of randomization and to assess clinical effects. All patients, clinicians (physicians, nurses, and respiratory care practitioners), and investigators will be blinded to treatment assignments.

### Inclusion Criteria Will Include the Following: [245]

1. Nonpregnant adults greater than 18 years of age.

2. Developed ALI/ARDS within the preceding 72 hours as defined as:

PaO2/FiO2<250, regardless of the amount of PEEP.
Bilateral infiltrates on frontal chest radiograph.
Pulmonary artery occlusion pressure <18 mm Hg when measured or no clinical evidence of left atrial hypertension via ultrasonographic or other modalities.

3. ALI/ARDS resulting from at least one of the following: pneumonia, aspiration pneumonitis, toxic gas inhalation, pulmonary contusion, acute pancreatitis, massive blood transfusion (including transfusion reactions), polytrauma trauma, elective or emergency major surgery, postpartum ALI.

4. FiO2 of 0.50-0.100 with any PEEP level and whom are mechanically ventilated.

### Exclusion Criteria

1. History of allergy or hypersensitivity to clostridial collagenase
2. History of congestive heart failure (this may be changed based on preliminary study results).
3. Pregnant.
4. Age less than 18 years of age (this may be changed based on preliminary study results).
5. Immunocompromised (Including: any active cancer of any time, currently on immunosuppressive medications, chemotherapy or radiation therapy).

Box 1 of [245] serves as a comparative example for inclusion and exclusion criteria that may be followed.

### Study Protocol

The study medications will be delivered via a VMN system or other applicable nebulizer systems[175]. During administration all patients will receive ventilatory support and will be continuously monitored including level of FiO2 requirements, arterial blood gas values (ABG), blood pressure as well as heart rate. Oxygenation and ventilation parameters including Vt, PEEP, compliance, and minute ventilation will be recorded at baseline and at 4 hours and 12 hours after initiation of nebulized agent for the first 24-hour period. After this 24-hour period these will be recorded every 12 hours thereafter for the 28-day study period. Chest radiographs will be obtained at baseline and daily. Complete blood cell counts, serum and BAL type III, type I procollagen peptide levels, Arterial Blood Gas, serum biochemistry values will be collected at baseline and daily thereafter. Adequate oxygenation will be defined as pulse oximetry oxygen saturation of 90% or more or PaO2 of 63 mm Hg or more. PaO2 will take precedence when both values are available. The above design is plausible as it has been previously described [245]. Additionally, respiratory support during the study will be titrated in a uniformed fashion as recommended by current clinical practice [245].

Box 2 of [245] serves as a comparative example of the guideline that will be employed during the study.

Specific definitions for the study will be as follows: 1) pneumonia: pulmonary infiltrates thought to be due to primary lung infection with fever, and/or leukocytosis and a sputum Gram stain with more than 25 white blood cells and less than 10 epithelial cells per low-power field; 2) aspiration event: clinical history compatible with aspiration of gastric material and/or witnessed aspiration; 3) pulmonary contusion: presence of lung infiltrates within 24 hours of inciting blunt trauma, 4) acute pancreatitis: syndrome characterized by increased serum amylase and/or lipase concentrations with one of the following: positive abdominal imaging consistent with pancreatitis or abdominal pain as determined by physical exam; 5) massive blood transfusion: more than 10 units of blood products within a 24 hour period; 6) postpartum acute lung injury: within 72-hours of delivery with no evidence of cardiac dysfunction; 7) acute lung injury associated with surgical procedure: patients whom underwent elective or emergent surgery with no other cause of acute lung injury identified. The above definitions are adapted from work of which has been previously described [245].

### Outcomes

Primary outcome measures of interest will be prospectively defined. The primary outcome of interest will be 28-day mortality.

Secondary outcomes of interest will be liberation from mechanical ventilatory support (defined as time to extubation), changes in lung compliance, oxygenation levels, amount of ventilatory support defined by: reduction in pressure support, Fio2 requirements and/or PEEP requirements, and adverse reactions to the administered agent. Additional biophysiological assessments not listed may also be conducted.

A comparative example of targeted outcomes after inhaled nitric oxide delivery for ARDS patients is provided in Table 2 and FIG. 2 of [245] and may be considered or followed.

### Assessing Dosing and Medication Safety

As mentioned previously single dose escalation study will be conducted [234] [235]. During administration of the study medication plasma, urine and/or BAL samples will be collected at different time points during the study period for inhalation for pharmacokinetic (PK) assessment [234] [235], as well as to measure both enzymatic components of nebulized Ccol and reactive antibodies to Ccol enzymes, as previously described [171, 246]. The initial administered dose/kg derived from preclinical animal models on a mg/kg basis will be administered with utilization of previously mentioned dose conversions methodologies. The dosing regimen will then be escalated after evaluation of all safety and PK data of the preceding dose level by a dedicated safety evaluation team [234] [235] [171]. Pharmacokinetic variables will be estimated using noncompartmental approaches [234] [171, 235]. Both enzymatic components as well as associated enzymes will be serially measured using validated enzyme-linked immunosorbent assays [234] [235] [171]. Samples including PK parameters will be assessed prior to medication administration at each dosage and after administration at 1 hour, 3 hours and 24 hours after inhalation, but may include additional measurement points. The treatment efficiency of the nebulized Ccol compound will be determined by assessing its performance efficiency[246] [186]. Toxicity and toxicokinetic profiles will be assessed as well as any reversibility of these toxicities[171, 246]. During each dosage point the enzymatic activity of nebulized Ccol may be alternatively assessed via enzymatic assay using Fluorescence Resonance Energy Transfer (FRET) as adapted from previously performed inhaled neutrophil elastase inhibitor studies[235, 247] [248]. The (fTHP) Förster resonance energy transfer triple-helical peptide substrate, which possesses a sequence: Gly-mep-Flp-(Gly-Pro-Hyp)4-Gly-Lys(Mca)-Thr-Gly-Pro-Leu-Gly-Pro-Pro-Gly-Lys(Dnp)-Ser-(Gly-Pro-Hyp)4-NH2], has melting point of (Tm) of 36.2° C. and is efficiently hydrolyzed by Ccol [247, 249]. The fTHP bacterial collagenase assay allows for rapid and specific assessment of enzyme activity toward triple helices[247, 249]. The efficacy, safety and adverse events will be compared between comparison groups and amongst different dosages[247, 250]. However, given that this medication has demonstrated low systemic exposure, no systematic toxicity and distribution to other end organs this may not necessarily be required.

FIGS. 2-4 of [247] serve as comparative examples of fluorescent substrate analysis for Clostridial collagenase activity assessment that may be performed.

FIG. 2 and Table 2 of [235] serve as a comparative example of previously performed single dose escalation study and analysis of PK parameters during inhaled elastase inhibitor delivery that may be performed or considered.

### Assessment of Cytokine Inflammatory Response, Cellular Characteristics and Protein Content During ARDS

Cytokine expression and lung lavage total protein concentration will be assessed at predetermined time points after administration of nebulized Ccol and control. These will include serum analysis of TNF-α, IL-1b, IL-6, IL-10, IFN-γ, MIP-2(Macrophage inflammatory protein) will be carried out using commercially available ELISA kits, quantitative PCR or expression arrays, amongst others [251] [252]. Amount of protein content as well as different cellular expression will be assessed as previously described [253].

Tables 3 and 4 [253] and Table 2 [252] provide comparative examples of results reporting for these specified assessments.

### Assessment of Nebulized Collagenase Activity & Effect on Lung Collagen Content

The effect of nebulized Ccol on lung collagen synthesis and subsequent degradation will be assessed. Both serum and BAL sample analysis will be utilized for relative collagen expression and by products of collagen degradation (procollagen type I and III) at predetermined set points after baseline level determination [15] [14] [127].

Expression of collagenase activity will also be monitored [254]. These will be compared amongst those in the control group and in those receiving the compound for any correlation during the study course.

FIG. 4 and the descriptions of [127] and Table 1, FIGS. 2 and 4 of [254] depict comparative examples of the methods and results for collagen content determination and collagenase activity in human subjects that may be considered or performed.

### REFERENCES

1. Egui Rojo, M. A., et al., Experience in the use of collagenase clostridium histolyticum in the management of Peyronie's disease: current data and future prospects. Therapeutic Advances in Urology, 2014. 6(5): p. 192-197.
2. Shi, L., et al., Degradation of human collagen isoforms by Clostridium collagenase and the effects of degradation products on cell migration. International Wound Journal, 2010. 7(2): p. 87-95.
3. Riley, K. N. and I. M. Herman, Collagenase promotes the cellular responses to injury and wound healing in vivo. Journal of burns and wounds, 2005. 4.
4. Shi, L. and D. Carson, Collagenase Santyl ointment: a selective agent for wound debridement. Journal of Wound Ostomy & Continence Nursing, 2009. 36(6S): p. S12-S16.
5. Ramundo, J. and M. Gray, Collagenase for enzymatic debridement: a systematic review. Journal of Wound Ostomy & Continence Nursing, 2009. 36(6S): p. S4-S11.
6. Das, A., et al., Novel mechanisms of Collagenase Santyl Ointment (CSO) in wound macrophage polarization and resolution of wound inflammation. Scientific reports, 2018. 8(1): p. 1696-1696.
7. Bellingan, G., The pulmonary physician in critical care•6: The pathogenesis of ALI/ARDS. Thorax, 2002. 57(6): p. 540-546.
8. González-López, A., et al., Inflammation and matrix remodeling during repair of ventilator-induced lung injury. American Journal of Physiology-Lung Cellular and Molecular Physiology, 2011. 301(4): p. L500-L509.
9. Ricou, B., et al., Matrix metalloproteinases and TIMP in acute respiratory distress syndrome. American journal of respiratory and critical care medicine, 1996. 154(2): p. 346-352.
10. Hamel, M. B., et al., Outcomes and cost-effectiveness of ventilator support and aggressive care for patients with acute respiratory failure due to pneumonia or acute respiratory distress syndrome. The American journal of medicine, 2000. 109(8): p. 614-620.
11. Bein, T., S. Weber-Carstens, and C. Apfelbacher, Long-term outcome after the acute respiratory distress syndrome: different from general critical illness? Current opinion in critical care, 2018. 24(1): p. 35-40.
12. Villar, J., et al., The ALIEN study: incidence and outcome of acute respiratory distress syndrome in the era of lung protective ventilation. Intensive care medicine, 2011. 37(12): p. 1932-1941.
13. Clark, J. G., et al., Type III procollagen peptide in the adult respiratory distress syndrome: association of increased peptide levels in bronchoalveolar lavage fluid with increased risk for death. Annals of internal medicine, 1995. 122(1): p. 17-23.
14. Chesnutt, A. N., et al., Early detection of type III procollagen peptide in acute lung injury: pathogenetic and prognostic significance. American journal of respiratory and critical care medicine, 1997. 156(3): p. 840-845.
15. Marshall, R. P., et al., Fibroproliferation occurs early in the acute respiratory distress syndrome and impacts on outcome. American journal of respiratory and critical care medicine, 2000. 162(5): p. 1783-1788.
16. Hayashi, T., et al., Immunohistochemical study of metalloproteinases and their tissue inhibitors in the lungs of patients with diffuse alveolar damage and idiopathic pulmonary fibrosis. The American journal of pathology, 1996. 149(4): p. 1241-1256.
17. Planus, E., et al., Role of collagenase in mediating in vitro alveolar epithelial wound repair. Journal of cell science, 1999. 112(2): p. 243-252.
18. Albaiceta, G. M., et al., Lack of matrix metalloproteinase-9 worsens ventilator-induced lung injury. American Journal of Physiology-Lung Cellular and Molecular Physiology, 2008. 294(3): p. L535-L543.
19. Parks, W. C., Matrix metalloproteinases in lung repair. European Respiratory Journal, 2003. 22(44 suppl): p. 36s-38s.
20. Miyoshi, S.-i., et al., Characterization of the hemorrhagic reaction caused by Vibrio vulnificus metalloprotease, a member of the thermolysin family. Infection and immunity, 1998. 66(10): p. 4851-4855.
21. French, M. F., A. Bhown, and H. E. Van Wart, Identification of Clostridium histolyticum collagenase hyperreactive sites in type I, II, and III collagens: lack of correlation with local triple helical stability. Journal of protein chemistry, 1992. 11(1): p. 83-97.
22. Panuncialman, J. and V. Falanga, The Science of Wound Bed Preparation. Surgical Clinics of North America, 2009. 89(3): p. 611-626.
23. Sheets, A. R., et al., Identification and Characterization of Novel Matrix-Derived Bioactive Peptides: A Role for Collagenase from Santyl® Ointment in Post-Debridement Wound Healing? PLOS ONE, 2016. 11(7): p. e0159598.
24. Toyoshima, T., et al., Collagen-binding domain of a Clostridium histolyticum collagenase exhibits a broad substrate spectrum both in vitro and in vivo. Connective tissue research, 2001. 42(4): p. 281-290.
25. Wind, J., et al., Epidemiology of acute lung injury and acute respiratory distress syndrome in The Netherlands: A survey. Respiratory medicine, 2007. 101(10): p. 2091-2098.
26. Luhr, O. R., et al., Incidence and mortality after acute respiratory failure and acute respiratory distress syndrome in Sweden, Denmark, and Iceland. The ARF Study Group. Am J Respir Crit Care Med, 1999. 159(6): p. 1849-61.
27. Cutts, S., et al., Adult respiratory distress syndrome. Annals of the Royal College of Surgeons of England, 2017. 99(1): p. 12-16.
28. Ranieri, V. M., et al., Acute respiratory distress syndrome: the Berlin Definition. Jama, 2012. 307(23): p. 2526-33.
29. Fanelli, V., et al., Acute respiratory distress syndrome: new definition, current and future therapeutic options. Journal of thoracic disease, 2013. 5(3): p. 326-334.
30. Brower, R. G., et al., Ventilation with lower tidal volumes as compared with traditional tidal volumes for acute lung injury and the acute respiratory distress syndrome. N Engl J Med, 2000. 342(18): p. 1301-8.
31. Eichacker, P. Q., et al., Meta-Analysis of Acute Lung Injury and Acute Respiratory Distress Syndrome Trials Testing Low Tidal Volumes. American Journal of Respiratory and Critical Care Medicine, 2002. 166(11): p. 1510-1514.
32. Pelosi, P., et al., Effects of the prone position on respiratory mechanics and gas exchange during acute lung injury. Am J Respir Crit Care Med, 1998. 157(2): p. 387-93.
33. Galvin, I. M., et al., Partial liquid ventilation for preventing death and morbidity in adults with acute lung injury and acute respiratory distress syndrome. Cochrane Database Syst Rev, 2013. 2013(7): p. Cd003707.
34. Khilnani, G. C. and V. Hadda, Corticosteroids and ARDS: A review of treatment and prevention evidence. Lung India: official organ of Indian Chest Society, 2011. 28(2): p. 114-119.
35. Hedenstierna, G. and J. B. Borges, Oxford Textbook of Critical Care, in Normal physiology of the respiratory system. 2016, Oxford University Press.
36. Chaudhry, R. and B. Bordoni, Anatomy, Thorax, Lungs, in StatPearls. 2022, StatPearls Publishing Copyright © 2022, StatPearls Publishing LLC.: Treasure Island (FL).
37. Haefeli-Bleuer, B. and E. R. Weibel, Morphometry of the human pulmonary acinus. Anat Rec, 1988. 220(4): p. 401-14.
38. Astrom, E., et al., Partitioning of dead space—a method and reference values in the awake human. European Respiratory Journal, 2000. 16(4): p. 659-664.
39. Brandt, J. P. and P. Mandiga, Histology, Alveolar Cells. 2020.
40. Hance, A. J. and R. G. Crystal, The connective tissue of lung. American Review of Respiratory Disease, 1975. 112(5): p. 657-711.
41. Raghu, G., et al., Extracellular matrix in normal and fibrotic human lungs. American Review of Respiratory Disease, 1985. 131(2): p. 281-289.
42. Burgstaller, G., et al., The instructive extracellular matrix of the lung: basic composition and alterations in chronic lung disease. European Respiratory Journal, 2017. 50(1): p. 1601805.
43. Suki, B. and J. H. T. Bates, Extracellular matrix mechanics in lung parenchymal diseases. Respiratory physiology & neurobiology, 2008. 163(1-3): p. 33-43.
44. Mithieux, S. M. and A. S. Weiss, Elastin, in Advances in Protein Chemistry. 2005, Academic Press. p. 437-461.
45. Brown, R. E., et al., Mechanical connections between elastin and collagen. Connect Tissue Res, 1994. 30(4): p. 295-308.
46. Levitzky, M. G., Chapter 2. Mechanics of Breathing, in Pulmonary Physiology, 8e. 2013, The McGraw-Hill Companies: New York, N.Y.
47. Frantz, C., K. M. Stewart, and V. M. Weaver, The extracellular matrix at a glance. Journal of cell science, 2010. 123(Pt 24): p. 4195-4200.
48. Suki, B., et al., Biomechanics of the lung parenchyma: critical roles of collagen and mechanical forces. J Appl Physiol (1985), 2005. 98(5): p. 1892-9.
49. Ng, A. W., A. Bidani, and T. A. Heming, Innate Host Defense of the Lung: Effects of Lung-lining Fluid pH. Lung, 2004. 182(5): p. 297-317.
50. Voynow, J. A., What does mucin have to do with lung disease? Paediatric respiratory reviews, 2002. 3(2): p. 98-103.
51. LeVine, A. M. and J. A. Whitsett, Pulmonary collectins and innate host defense of the lung. Microbes and Infection, 2001. 3(2): p. 161-166.
52. Sou, T. and C. A. S. Bergström, Contemporary Formulation Development for Inhaled Pharmaceuticals. Journal of Pharmaceutical Sciences, 2021. 110(1): p. 66-86.
53. Fröhlich, E., et al., Measurements of Deposition, Lung Surface Area and Lung Fluid for Simulation of Inhaled Compounds. Frontiers in Pharmacology, 2016. 7.
54. Johnson, M. D., et al., Alveolar epithelial type I cells contain transport proteins and transport sodium, supporting an active role for type I cells in regulation of lung liquid homeostasis. Proceedings of the National Academy of Sciences of the United States of America, 2002. 99(4): p. 1966-1971.
55. Crandall, E. D. and M. A. Matthay, Alveolar epithelial transport. Basic science to clinical medicine. Am J Respir Crit Care Med, 2001. 163(4): p. 1021-9.
56. Lubman, R. L. and E. D. Crandall, Polarized distribution of Na (+)-H+ antiport activity in rat alveolar epithelial cells. American Journal of Physiology-Lung Cellular and Molecular Physiology, 1994. 266(2): p. L138-L147.
57. Lubman, R. L., et al., Cl (−)HCO3-exchanger isoform AE2 is restricted to the basolateral surface of alveolar epithelial cell monolayers. American journal of respiratory cell and molecular biology, 1995. 12(2): p. 211-219.
58. Lubman, R. L., D. C. Chao, and E. D. Crandall, Basolateral localization of Na+-HCO3-cotransporter activity in alveolar epithelial cells. Respiration Physiology, 1995. 100(1): p. 15-24.
59. Nielson, D. W., J. Goerke, and J. A. Clements, Alveolar subphase pH in the lungs of anesthetized rabbits. Proceedings of the National Academy of Sciences of the United States of America, 1981. 78(11): p. 7119-7123.
60. Xue, M. and C. J. Jackson, Extracellular Matrix Reorganization During Wound Healing and Its Impact on Abnormal Scarring. Advances in wound care, 2015. 4(3): p. 119-136.
61. Petreaca, M. L., et al., Transactivation of vascular endothelial growth factor receptor-2 by interleukin-8 (IL-8/CXCL8) is required for IL-8/CXCL8-induced endothelial permeability. Molecular biology of the cell, 2007. 18(12): p. 5014-5023.
62. Suzuki-Inoue, K., [Activation and inhibitory mechanisms of blood platelets]. Nihon Rinsho, 2014. 72(7): p. 1212-7.
63. Schultz, G. S. and A. Wysocki, Interactions between extracellular matrix and growth factors in wound healing. Wound Repair Regen, 2009. 17(2): p. 153-62.
64. Marin, V., et al., The IL-6-soluble IL-6Ralpha autocrine loop of endothelial activation as an intermediate between acute and chronic inflammation: an experimental model involving thrombin. J Immunol, 2001. 167(6): p. 3435-42.
65. Zgheib, C., J. Xu, and K. W. Liechty, Targeting Inflammatory Cytokines and Extracellular Matrix Composition to Promote Wound Regeneration. Advances in wound care, 2014. 3(4): p. 344-355.
66. Simpson, D. M. and R. Ross, The neutrophilic leukocyte in wound repair a study with antineutrophil serum. J Clin Invest, 1972. 51(8): p. 2009-23.
67. Brizzi, M. F., G. Tarone, and P. Defilippi, Extracellular matrix, integrins, and growth factors as tailors of the stem cell niche. Current Opinion in Cell Biology, 2012. 24(5): p. 645-651.
68. Singer, A. J. and R. A. Clark, Cutaneous wound healing. New England journal of medicine, 1999. 341(10): p. 738-746.
69. Mathew-Steiner, S. S., S. Roy, and C. K. Sen, Collagen in Wound Healing. Bioengineering, 2021. 8(5): p. 63.
70. Grotendorst, G. R., et al., Stimulation of granulation tissue formation by platelet-derived growth factor in normal and diabetic rats. J Clin Invest, 1985. 76(6): p. 2323-9.
71. Rapraeger, A. C., A. Krufka, and B. B. Olwin, Requirement of heparan sulfate for bFGF-mediated fibroblast growth and myoblast differentiation. Science, 1991. 252(5013): p. 1705-8.
72. Ortéga, N., F. E. L'Faqihi, and J. Plouët, Control of vascular endothelial growth factor angiogenic activity by the extracellular matrix. Biol Cell, 1998. 90(5): p. 381-90.
73. Edwards, D. R., et al., Transforming growth factor beta modulates the expression of collagenase and metalloproteinase inhibitor. Embo j, 1987. 6(7): p. 1899-904.
74. Mathew-Steiner, S. S., S. Roy, and C. K. Sen, Collagen in Wound Healing. Bioengineering (Basel, Switzerland), 2021. 8(5): p. 63.
75. Egeblad, M., M. G. Rasch, and V. M. Weaver, Dynamic interplay between the collagen scaffold and tumor evolution. Current Opinion in Cell Biology, 2010. 22(5): p. 697-706.
76. Profyris, C., C. Tziotzios, and I. Do Vale, Cutaneous scarring: Pathophysiology, molecular mechanisms, and scar reduction therapeutics: Part I. The molecular basis of scar formation. Journal of the American Academy of Dermatology, 2012. 66(1): p. 1-10.
77. Ricard-Blum, S., The collagen family. Cold Spring Harbor perspectives in biology, 2011. 3(1): p. a004978-a004978.
78. Zhang, Y.-Z., et al., Diversity, structures, and collagen-degrading mechanisms of bacterial collagenolytic proteases. Applied and environmental microbiology, 2015. 81(18): p. 6098-6107.
79. Kadler, K. E., et al., Collagen fibril formation. The Biochemical journal, 1996. 316 (Pt 1)(Pt 1): p. 1-11.
80. Yamauchi, M. and M. Sricholpech, Lysine post-translational modifications of collagen. Essays in biochemistry, 2012. 52: p. 113-133.
81. Silver, F. H., J. W. Freeman, and G. P. Seehra, Collagen self-assembly and the development of tendon mechanical properties. J Biomech, 2003. 36(10): p. 1529-53.
82. Nagase, H., R. Visse, and G. Murphy, Structure and function of matrix metalloproteinases and TIMPs. Cardiovasc Res, 2006. 69(3): p. 562-73.
83. Knapinska, A. and G. B. Fields, Chemical biology for understanding matrix metalloproteinase function. Chembiochem: a European journal of chemical biology, 2012. 13(14): p. 2002-2020.
84. Cui, N., M. Hu, and R. A. Khalil, Biochemical and biological attributes of matrix metalloproteinases. Progress in molecular biology and translational science, 2017. 147: p. 1-73.
85. Murphy, G. and V. Knäuper, Relating matrix metalloproteinase structure to function: why the “hemopexin” domain? Matrix Biol, 1997. 15(8-9): p. 511-8.
86. Chung, L., et al., Collagenase unwinds triple-helical collagen prior to peptide bond hydrolysis. Embo j, 2004. 23(15): p. 3020-30.
87. Van Doren, S. R., Matrix metalloproteinase interactions with collagen and elastin. Matrix Biology, 2015. 44-46: p. 224-231.
88. Houghton, A. M., M. Mouded, and S. D. Shapiro, Consequences of elastolysis, in Extracellular matrix degradation. 2011, Springer. p. 217-249.
89. Jackson, C. J., et al., Activated protein C prevents inflammation yet stimulates angiogenesis to promote cutaneous wound healing. Wound Repair Regen, 2005. 13(3): p. 284-94.
90. Szpaderska, A. M. and L. A. DiPietro, Inflammation in surgical wound healing: friend or foe? Surgery, 2005. 137(5): p. 571-3.
91. Khanna, S., et al., Macrophage dysfunction impairs resolution of inflammation in the wounds of diabetic mice. PLoS One, 2010. 5(3): p. e9539.
92. Hesketh, M., et al., Macrophage Phenotypes Regulate Scar Formation and Chronic Wound Healing. Int J Mol Sci, 2017. 18(7).
93. Verhaegen, P. D., et al., Differences in collagen architecture between keloid, hypertrophic scar, normotrophic scar, and normal skin: An objective histopathological analysis. Wound Repair Regen, 2009. 17(5): p. 649-56.
94. Slemp, A. E. and R. E. Kirschner, Keloids and scars: a review of keloids and scars, their pathogenesis, risk factors, and management. Current opinion in pediatrics, 2006. 18(4): p. 396-402.
95. Lee, D. E., et al., High-mobility Group Box Protein-1, Matrix Metalloproteinases, and Vitamin D in Keloids and Hypertrophic Scars. Plastic and reconstructive surgery. Global open, 2015. 3(6): p. e425-e425.
96. Ghahary, A., et al., Collagenase production is lower in post-burn hypertrophic scar fibroblasts than in normal fibroblasts and is reduced by insulin-like growth factor-1. J Invest Dermatol, 1996. 106(3): p. 476-81.
97. Arakawa, M., et al., Reduced collagenase gene expression in fibroblasts from hypertrophic scar tissue. British journal of dermatology (1951), 1996. 134(5): p. 863-868.
98. Diegelmann, R. F., I. K. Cohen, and B. J. McCoy, Growth kinetics and collagen synthesis of normal skin, normal scar and keloid fibroblasts in vitro. J Cell Physiol, 1979. 98(2): p. 341-6.
99. Murrell, G. A., M. J. Francis, and L. Bromley, The collagen changes of Dupuytren's contracture. J Hand Surg Br, 1991. 16(3): p. 263-6.
100. Brickley-Parsons, D., et al., Biochemical changes in the collagen of the palmar fascia in patients with Dupuytren's disease. J Bone Joint Surg Am, 1981. 63(5): p. 787-97.
101. Melling, M., et al., Changes of biochemical and biomechanical properties in Dupuytren disease. Arch Pathol Lab Med, 2000. 124(9): p. 1275-81.
102. Syed, F., et al., In vitro study of novel collagenase (XIAFLEX®) on Dupuytren's disease fibroblasts displays unique drug related properties. PLoS One, 2012. 7(2): p. e31430.
103. Kaufman-Janette, J., et al., Collagenase Clostridium Histolyticum-aaes for the Treatment of Cellulite in Women: Results From Two Phase 3 Randomized, Placebo-Controlled Trials. Dermatol Surg, 2021. 47(5): p. 649-656.
104. Rudolph, C., et al., Structural Gender Dimorphism and the Biomechanics of the Gluteal Subcutaneous Tissue: Implications for the Pathophysiology of Cellulite. Plast Reconstr Surg, 2019. 143(4): p. 1077-1086.
105. Amariei, D. E., et al., Combined Pulmonary Fibrosis and Emphysema: Pulmonary Function Testing and a Pathophysiology Perspective. Medicina, 2019. 55(9): p. 580.
106. Tuder, R. M. and I. Petrache, Pathogenesis of chronic obstructive pulmonary disease. The Journal of clinical investigation, 2012. 122(8): p. 2749-2755.
107. Goldklang, M. and R. Stockley, Pathophysiology of Emphysema and Implications. Chronic obstructive pulmonary diseases, 2016. 3(1): p. 454-458.
108. Imai, K., et al., Human collagenase (matrix metalloproteinase-1) expression in the lungs of patients with emphysema. American journal of respiratory and critical care medicine, 2001. 163(3): p. 786-791.
109. Ohnishi, K., et al., Matrix metalloproteinase-mediated extracellular matrix protein degradation in human pulmonary emphysema. Laboratory investigation; a journal of technical methods and pathology, 1998. 78(9): p. 1077-1087.
110. Betsuyaku, T., et al., Neutrophil granule proteins in bronchoalveolar lavage fluid from subjects with subclinical emphysema. Am J Respir Crit Care Med, 1999. 159(6): p. 1985-91.
111. Finlay, G. A., et al., Elevated levels of matrix metalloproteinases in bronchoalveolar lavage fluid of emphysematous patients. Thorax, 1997. 52(6): p. 502-6.
112. Shapiro, S. D., Elastolytic metalloproteinases produced by human mononuclear phagocytes. Potential roles in destructive lung disease. Am J Respir Crit Care Med, 1994. 150(6 Pt 2): p. S160-4.
113. Kononov, S., et al., Roles of mechanical forces and collagen failure in the development of elastase-induced emphysema. Am J Respir Crit Care Med, 2001. 164(10 Pt 1): p. 1920-6.
114. Montaño, M., et al., Lung Collagenase Inhibitors and Spontaneous and Latent Collagenase Activity in Idiopathic Pulmonary Fibrosis and Hypersensitivity Pneumonitis. Chest, 1989. 96(5): p. 1115-1119.
115. Dolhnikoff, M., T. Mauad, and M. S. Ludwig, Extracellular matrix and oscillatory mechanics of rat lung parenchyma in bleomycin-induced fibrosis. Am J Respir Crit Care Med, 1999. 160(5 Pt 1): p. 1750-7.
116. Wolters, P. J., H. R. Collard, and K. D. Jones, Pathogenesis of idiopathic pulmonary fibrosis. Annu Rev Pathol, 2014. 9: p. 157-79.
117. Noble, P. W., C. E. Barkauskas, and D. Jiang, Pulmonary fibrosis: patterns and perpetrators. J Clin Invest, 2012. 122(8): p. 2756-62.
118. Decaris, M. L., et al., Proteomic analysis of altered extracellular matrix turnover in bleomycin-induced pulmonary fibrosis. Mol Cell Proteomics, 2014. 13(7): p. 1741-52.
119. Frederick, R. E., et al., Clostridium Collagenase Impact on Zone of Stasis Stabilization and Transition to Healthy Tissue in Burns. Int J Mol Sci, 2021. 22(16).
120. Kligerman, S. J., T. J. Franks, and J. R. Galvin, From the radiologic pathology archives: organization and fibrosis as a response to lung injury in diffuse alveolar damage, organizing pneumonia, and acute fibrinous and organizing pneumonia. Radiographics, 2013. 33(7): p. 1951-1975.
121. Spadaro, S., et al., Biomarkers for Acute Respiratory Distress syndrome and prospects for personalised medicine. Journal of inflammation (London, England), 2019. 16: p. 1-1.
122. Matthay, M. A., L. B. Ware, and G. A. Zimmerman, The acute respiratory distress syndrome. The Journal of clinical investigation, 2012. 122(8): p. 2731-2740.
123. Tremblay, L., et al., Injurious ventilatory strategies increase cytokines and c-fos m-RNA expression in an isolated rat lung model. The Journal of clinical investigation, 1997. 99(5): p. 944-952.
124. Suki, B., K. R. Lutchen, and E. P. Ingenito, On the progressive nature of emphysema: roles of proteases, inflammation, and mechanical forces. Am J Respir Crit Care Med, 2003. 168(5): p. 516-21.
125. Maksym, G. N. and J. H. Bates, A distributed nonlinear model of lung tissue elasticity. J Appl Physiol (1985), 1997. 82(1): p. 32-41.
126. Rocco, P. R., et al., Lung tissue mechanics and extracellular matrix remodeling in acute lung injury. American journal of respiratory and critical care medicine, 2001. 164(6): p. 1067-1071.
127. Armstrong, L., et al., Changes in collagen turnover in early acute respiratory distress syndrome. Am J Respir Crit Care Med, 1999. 160(6): p. 1910-5.
128. Thille, A. W., et al., Chronology of histological lesions in acute respiratory distress syndrome with diffuse alveolar damage: a prospective cohort study of clinical autopsies. The lancet Respiratory medicine, 2013. 1(5): p. 395-401.
129. Snyder, L. S., et al., Acute lung injury. Pathogenesis of intraalveolar fibrosis. The Journal of clinical investigation, 1991. 88(2): p. 663-673.
130. Angele, P., et al., Influence of different collagen species on physico-chemical properties of crosslinked collagen matrices. Biomaterials, 2004. 25(14): p. 2831-41
131. Buisson, A. C., et al., Gelatinase B is involved in the in vitro wound repair of human respiratory epithelium. Journal of cellular physiology, 1996. 166(2): p. 413-426.
132. Castro, C. Y. ARDS and diffuse alveolar damage: a pathologist's perspective. in Seminars in thoracic and cardiovascular surgery. 2006. Elsevier.
133. Zapol, W. M., et al., Pulmonary fibrosis in severe acute respiratory failure. American Review of Respiratory Disease, 1979. 119(4): p. 547-554.
134. Zapol, W. M., et al., Pulmonary fibrosis in severe acute respiratory failure. Am Rev Respir Dis, 1979. 119(4): p. 547-54.
135. Boesing, C., et al., Procollagen I and III as Prognostic Markers in Patients Treated with Extracorporeal Membrane Oxygenation: A Prospective Observational Study. Journal of clinical medicine, 2021. 10(16): p. 3686.
136. Jabaudon, M., et al., Soluble Receptor for Advanced Glycation End-Products Predicts Impaired Alveolar Fluid Clearance in Acute Respiratory Distress Syndrome. Am J Respir Crit Care Med, 2015. 192(2): p. 191-9.
137. Sato, H., et al., KL-6 levels are elevated in plasma from patients with acute respiratory distress syndrome. Eur Respir J, 2004. 23(1): p. 142-5.
138. Koh, H., et al., Vascular endothelial growth factor in epithelial lining fluid of patients with acute respiratory distress syndrome. Respirology, 2008. 13(2): p. 281-4.
139. Ware, L. B., et al., Prognostic and pathogenetic value of combining clinical and biochemical indices in patients with acute lung injury. Chest, 2010. 137(2): p. 288-96.
140. Terpstra, M. L., et al., Plasma biomarkers for acute respiratory distress syndrome: a systematic review and meta-analysis*. Crit Care Med, 2014. 42(3): p. 691-700.
141. Determann, R. M., et al., Plasma levels of surfactant protein D and KL-6 for evaluation of lung injury in critically ill mechanically ventilated patients. BMC Pulm Med, 2010. 10: p. 6.
142. Gertler, R., Respiratory Mechanics. Anesthesiology clinics, 2021. 39(3): p. 415-440.
143. Grinnan, D. C. and J. D. Truwit, Clinical review: Respiratory mechanics in spontaneous and assisted ventilation. Critical Care, 2005. 9(5): p. 472.
144. de Chazal, I. and R. D. Hubmayr, Novel aspects of pulmonary mechanics in intensive care. Br J Anaesth, 2003. 91(1): p. 81-91.
145. Grassino, A., C. Roussos, and P. Macklem, Static properties of the lung and chest wall. The lung: scientific foundations, 1997. 2: p. 1187-202.
146. Faffe, D. S. and W. A. Zin, Lung parenchymal mechanics in health and disease. Physiological reviews, 2009. 89(3): p. 759-775.
147. Giancotti, F. G. and E. Ruoslahti, Integrin signaling. Science, 1999. 285(5430): p. 1028-32.
148. Wagner, P. D., The physiological basis of pulmonary gas exchange: implications for clinical interpretation of arterial blood gases. European Respiratory Journal, 2015. 45(1): p. 227-243.
149. Eckhard, U., et al., Structure of collagenase G reveals a chew-and-digest mechanism of bacterial collagenolysis. Nature structural & molecular biology, 2011. 18(10): p. 1109-1114.
150. Eckhard, U., E. Schönauer, and H. Brandstetter, Structural basis for activity regulation and substrate preference of clostridial collagenases G, H, and T. Journal of Biological Chemistry, 2013. 288(28): p. 20184-20194.
151. Yoshihara, K., et al., Cloning and nucleotide sequence analysis of the colH gene from Clostridium histolyticum encoding a collagenase and a gelatinase. Journal of Bacteriology, 1994. 176(21): p. 6489-6496.
152. French, M. F., K. A. Mookhtiar, and H. E. Van Wart, Limited proteolysis of type I collagen at hyperreactive sites by class I and II Clostridium histolyticum collagenases: complementary digestion patterns. Biochemistry, 1987. 26(3): p. 681-687.
153. Breite, A. G., R. C. McCarthy, and F. E. Dwulet, Characterization and functional assessment of Clostridium histolyticum class I (C1) collagenases and the synergistic degradation of native collagen in enzyme mixtures containing class II (C2) collagenase. Transplant Proc, 2011. 43(9): p. 3171-5.
154. Mookhtiar, K. A., D. R. Steinbrink, and H. E. Van Wart, Mode of hydrolysis of collagen-like peptides by class I and class II Clostridium histolyticum collagenases: evidence for both endopeptidase and tripeptidylcarboxypeptidase activities. Biochemistry, 1985. 24(23): p. 6527-33.
155. Van Wart, H. E. and D. R. Steinbrink, Complementary substrate specificities of class I and class II collagenases from Clostridium histolyticum. Biochemistry, 1985. 24(23): p. 6520-6.
156. Berisio, R., et al., Crystal structure of a collagen-like polypeptide with repeating sequence Pro-Hyp-Gly at 1.4 A resolution: implications for collagen hydration. Biopolymers, 2000. 56(1): p. 8-13.
157. Breite, A. G., R. C. McCarthy, and F. E. Dwulet, Characterization and Functional Assessment of Clostridium Histolyticum Class I (C1) Collagenases and the Synergistic Degradation of Native Collagen in Enzyme Mixtures Containing Class II (C2) Collagenase. Transplantation Proceedings, 2011. 43(9): p. 3171-3175.
158. Mandl, I., S. Keller, and J. Manahan, Multiplicity of Clostridium histolyticum Collagenases. Biochemistry (Easton), 1964. 3(11): p. 1737-1741.
159. Hu, Y., et al., Rapid determination of substrate specificity of Clostridium histolyticum beta-collagenase using an immobilized peptide library. J Biol Chem, 2002. 277(10): p. 8366-71.
160. Manka, S. W., et al., Structural insights into triple-helical collagen cleavage by matrix metalloproteinase 1. Proc Natl Acad Sci USA, 2012. 109(31): p. 12461-6.
161. Galperin, R. C., et al., Anti-inflammatory Effects of Clostridial Collagenase: Results from In Vitro and Clinical Studies. Journal of the American Podiatric Medical Association, 2015. 105(6): p. 509-519.
162. Tallis, A., et al., Clinical and Economic Assessment of Diabetic Foot Ulcer Debridement with Collagenase: Results of a Randomized Controlled Study. Clinical Therapeutics, 2013. 35(11): p. 1805-1820.
163. Carter, M. J., et al., Cost effectiveness of adding clostridial collagenase ointment to selective debridement in individuals with stage IV pressure ulcers. Journal of Medical Economics, 2017. 20(3): p. 253-265.
164. Jia, S., et al., The effects of collagenase ointment on the prevention of hypertrophic scarring in a rabbit ear scarring model: a pilot study. Wounds, 2011. 23(6): p. 160-5.
165. Ralph, D. J., A. Abdel Raheem, and G. Liu, Treatment of Peyronie's Disease With Collagenase Clostridium histolyticum and Vacuum Therapy: A Randomized, Open-Label Pilot Study. The Journal of Sexual Medicine, 2017. 14(11): p. 1430-1437.
166. Peimer, C. A. M. D., et al., Dupuytren Contracture Recurrence Following Treatment With Collagenase Clostridium Histolyticum (CORDLESS [Collagenase Option for Reduction of Dupuytren Long-Term Evaluation of Safety Study]): 5-Year Data. The Journal of hand surgery (American ed.), 2015. 40(8): p. 1597-1605.
167. Aldemir, M., et al., Effectiveness of collagenase in the treatment of sacrococcygeal pilonidal sinus disease. Surg Today, 2003. 33(2): p. 106-9.
168. Patry, J. and V. Blanchette, Enzymatic debridement with collagenase in wounds and ulcers: a systematic review and meta-analysis. International wound journal, 2017. 14(6): p. 1055-1065.
169. Pham, C. H., et al., The role of collagenase ointment in acute burns: a systematic review and meta-analysis. Journal of wound care, 2019. 28(Sup2): p. S9-S15.
170. Smith & Nephew, I., Santyl (Collagenase, package insert). 2016: Fort Worth, Tex.
171. Bhatia, A. C., et al., Human Pharmacokinetics and Safety of Subcutaneous Collagenase Clostridium Histolyticum in Women. J Drugs Dermatol, 2020. 19(9): p. 852-856.
172. Pharma, E., Xiaflex (collagenase histolyticum, package insert). 2018: Malver, Pa.
173. Fratzl, P. and A. Daxer, Structural transformation of collagen fibrils in corneal stroma during drying. An x-ray scattering study. Biophys J, 1993. 64(4): p. 1210-4.
174. Snowden, J. M., The stabilization of in vivo assembled collagen fibrils by proteoglycans/glycosaminoglycans. Biochimica et biophysica acta, 1982. 703 1: p. 21-5.
175. McCarthy, S. D., H. E. González, and B. D. Higgins, Future trends in nebulized therapies for pulmonary disease. Journal of Personalized Medicine, 2020. 10(2): p. 37.
176. Tashkin, D. P., A review of nebulized drug delivery in COPD. International journal of chronic obstructive pulmonary disease, 2016. 11: p. 2585-2596.
177. Nelson, H., et al., Comparative Efficacy and Safety of Albuterol Sulfate Spiros Inhaler and Albuterol Metered-Dose Inhaler in Asthma. Chest, 1999. 115(2): p. 329-335.
178. Anderson, S., et al., Inhaled Medicines: Past, Present, and Future. Pharmacological Reviews, 2022. 74(1): p. 48-118.
179. Sigma Aldrich, I., Collagenase from Clostridium histolyticum. Product information. 2007-2008: Saint Louis, Mo.
180. Wolfe, H. R., et al., Evaluation of alternative diluents for clinical use of collagenase clostridium histolyticum (CCH-aaes). J Cosmet Dermatol, 2021. 20(6): p. 1643-1647.
181. Hertel, S., Pulmonary delivery of pharmaceutical proteins by means of vibrating mesh nebulization. 2014, Imu.
182. Jovanovic, A., et al., The influence of metal salts, surfactants, and wound care products on enzymatic activity of collagenase, the wound debriding enzyme. Wounds, 2012. 24(9): p. 242.
183. Forde, É., et al., Vibrating mesh nebulisation of pro-antimicrobial peptides for use in cystic fibrosis. Pharmaceutics, 2019. 11(5): p. 239.
184. Fröhlich, E. and S. Salar-Behzadi, Oral inhalation for delivery of proteins and peptides to the lungs. European Journal of Pharmaceutics and Biopharmaceutics, 2021. 163: p. 198-211.
185. Ari, A., et al., Influence of nebulizer type, position, and bias flow on aerosol drug delivery in simulated pediatric and adult lung models during mechanical ventilation. Respir Care, 2010. 55(7): p. 845-51.
186. Hess, D. R., Aerosol delivery devices in the treatment of asthma. Respiratory care, 2008. 53(6): p. 699-725.
187. Liu, C.-Y., et al., Size Distribution of Colistin Delivery by Different Type Nebulizers and Concentrations During Mechanical Ventilation. Pharmaceutics, 2019. 11(9): p. 459.
188. Sweeney, L., et al., Effective nebulization of interferon-γ using a novel vibrating mesh. Respiratory Research, 2019. 20(1): p. 1-11.
189. Ari, A., Aerosol therapy in pulmonary critical care. Respiratory Care, 2015. 60(6): p. 858-879.
190. Johnson, J. C., et al., Aerosol delivery of recombinant human DNase I: in vitro comparison of a vibrating-mesh nebulizer with a jet nebulizer. Respiratory care, 2008. 53(12): p. 1703-1708.
191. Pritchard, J. N., et al., Mesh nebulizers have become the first choice for new nebulized pharmaceutical drug developments. Ther Deliv, 2018. 9(2): p. 121-136.
192. Longest, W., B. Spence, and M. Hindle, Devices for Improved Delivery of Nebulized Pharmaceutical Aerosols to the Lungs. J Aerosol Med Pulm Drug Deliv, 2019. 32(5): p. 317-339.
193. Johanson, W. G., Jr. and A. K. Pierce, Effects of elastase, collagenase, and papain on structure and function of rat lungs in vitro. The Journal of clinical investigation, 1972. 51(2): p. 288-293.
194. Yi, E., et al., Mechanical Forces Accelerate Collagen Digestion by Bacterial Collagenase in Lung Tissue Strips. Frontiers in Physiology, 2016. 7.
195. Yoshihara, K., et al., Cloning and nucleotide sequence analysis of the colH gene from Clostridium histolyticum encoding a collagenase and a gelatinase. J Bacteriol, 1994. 176(21): p. 6489-96.
196. International, E., Qwo (clostrdial collagenase, for injection, product information). 2020: Malvern, Pa.
197. Hoorn, E. J., Intravenous fluids: balancing solutions. Journal of nephrology, 2017. 30(4): p. 485-492.
198. Klagsbrun, M., Large-scale preparation of chondrocytes. Methods Enzymol, 1979. 58: p. 560-4.
199. Mookhtiar, K. A., S. K. Mallya, and H. E. Van Wart, Properties of radiolabeled type I, II, and III collagens related to their use as substrates in collagenase assays. Anal Biochem, 1986. 158(2): p. 322-33.
200. Bassleer, C., et al., Human chondrocytes in tridimensional culture. In Vitro Cellular & Developmental Biology, 1986. 22(3): p. 113-119.
201. Angleton, E. L. and H. E. Van Wart, Preparation and reconstitution with divalent metal ions of class I and class II Clostridium histolyticum apocollagenases. Biochemistry, 1988. 27(19): p. 7406-7412.
202. Hoppe, I. J., H. Brandstetter, and E. Schönauer, Biochemical characterisation of a collagenase from Bacillus cereus strain Q1. Scientific Reports, 2021. 11(1): p. 4187.
203. Mandl, I., J. D. Maclennan, and E. L. Howes, Isolation and characterization of proteinase and collagenase from Cl. histolyticum. J Clin Invest, 1953. 32(12): p. 1323-9.
204. Stack, M. S. and R. D. Gray, Comparison of vertebrate collagenase and gelatinase using a new fluorogenic substrate peptide. J Biol Chem, 1989. 264(8): p. 4277-81.
205. Steinbrink, D. R., M. D. Bond, and H. E. Van Wart, Substrate specificity of beta-collagenase from Clostridium histolyticum. Journal of Biological Chemistry, 1985. 260(5): p. 2771-2776.
206. Jackson, R. J., D. V. Lim, and M. L. Dao, Identification and Analysis of a Collagenolytic Activity in Streptococcus mutans. Current Microbiology, 1997. 34(1): p. 49-54.
207. Gray, R. D. and H. H. Saneii, Characterization of vertebrate collagenase activity by high-performance liquid chromatography using a synthetic substrate. Anal Biochem, 1982. 120(2): p. 339-46.
208. Van Wart, H. E. and D. R. Steinbrink, A continuous spectrophotometric assay for Clostridium histolyticum collagenase. Analytical Biochemistry, 1981. 113(2): p. 356-365.
209. Wuensch, E. and H. G. Heidrich, [ON THE QUANTITATIVE DETERMINATION OF COLLAGENASE]. Hoppe Seylers Z Physiol Chem, 1963. 333: p. 149-51.
210. Kojima, K., et al., A new and highly sensitive fluorescence assay for collagenase-like peptidase activity. Anal Biochem, 1979. 100(1): p. 43-50.
211. Darlak, K., et al., Thiol-based inhibitors of mammalian collagenase. Substituted amide and peptide derivatives of the leucine analogue, 2-[(R,S)-mercaptomethyl]-4-methylpentanoic acid. Journal of Biological Chemistry, 1990. 265(9): p. 5199-5205.
212. Wilson, J. J., et al., A bacterial collagen-binding domain with novel calcium-binding motif controls domain orientation. The EMBO journal, 2003. 22(8): p. 1743-1752.
213. Angleton, E. L. and H. E. Van Wart, Preparation and reconstitution with divalent metal ions of class I and class II Clostridium histolyticum apocollagenases. Biochemistry, 1988. 27(19): p. 7406-12.
214. Clift, C. L., et al., Collagen fiber regulation in human pediatric aortic valve development and disease. Sci Rep, 2021. 11(1): p. 9751.
215. Roda, G., et al., Ripe and Raw Pu-Erh Tea: LC-MS Profiling, Antioxidant Capacity and Enzyme Inhibition Activities of Aqueous and Hydro-Alcoholic Extracts. Molecules, 2019. 24(3).
216. BioVision, Clostrdial Collagenase Colorimetric Activity Assay, product information. 2020: Milpitas, Calif.
217. Seifter, S., et al., Studies on collagen. II. Properties of purified collagenase and its inhibition. J Biol Chem, 1959. 234(2): p. 285-93.
218. Inamori, Y., et al., Antimicrobial activity and metalloprotease inhibition of hinokitiol-related compounds, the constituents of Thujopsis dolabrata S. and Z. hondai MAK. Biol Pharm Bull, 1999. 22(9): p. 990-3.
219. Seglen, P. O., Preparation of isolated rat liver cells. Methods Cell Biol, 1976. 13: p. 29-83.
220. Phillips, J. E., X. Zhang, and J. A. Johnston, Dry Powder and Nebulized Aerosol Inhalation of Pharmaceuticals Delivered to Mice Using a Nose-only Exposure System. Journal of visualized experiments: JoVE, 2017(122): p. 55454.
221. Phillips, J. E., Inhaled efficacious dose translation from rodent to human: A retrospective analysis of clinical standards for respiratory diseases. Pharmacol Ther, 2017. 178: p. 141-147.
222. Patil-Gadhe, A. A., et al., Rifapentine-proliposomes for inhalation: in vitro and in vivo toxicity. Toxicology international, 2014. 21(3): p. 275-282.
223. Muñoz-Barrutia, A., et al., Quantification of lung damage in an elastase-induced mouse model of emphysema. International journal of biomedical imaging, 2012. 2012: p. 734734-734734.
224. Liu, X., et al., The development of models for the evaluation of pulmonary drug disposition. Expert Opin Drug Metab Toxicol, 2013. 9(4): p. 487-505.
225. Bosquillon, C., et al., A Comparison of Drug Transport in Pulmonary Absorption Models: Isolated Perfused rat Lungs, Respiratory Epithelial Cell Lines and Primary Cell Culture. Pharmaceutical research, 2017. 34(12): p. 2532-2540.
226. Oldham, M. J., R. F. Phalen, and T. Budiman, Comparison of Predicted and Experimentally Measured Aerosol Deposition Efficiency in BALB/C Mice in a New Nose-Only Exposure System. Aerosol Science and Technology, 2009. 43(10): p. 970-977.
227. Ménache, M. G., F. J. Miller, and O. G. Raabe, Particle inhalability curves for humans and small laboratory animals. Ann Occup Hyg, 1995. 39(3): p. 317-28.
228. MacLoughlin, R. J., et al., Optimized aerosol delivery to a mechanically ventilated rodent. J Aerosol Med Pulm Drug Deliv, 2009. 22(4): p. 323-32.
229. Sakagami, M., In vivo, in vitro and ex vivo models to assess pulmonary absorption and disposition of inhaled therapeutics for systemic delivery. Adv Drug Deliv Rev, 2006. 58(9-10): p. 1030-60.
230. Miller, F. J., et al., Improvements and additions to the Multiple Path Particle Dosimetry model. Journal of Aerosol Science, 2016. 99: p. 14-26.
231. Ji, J. H. and I. J. Yu, Estimation of human equivalent exposure from rat inhalation toxicity study of silver nanoparticles using multi-path particle dosimetry model. Toxicology Research, 2012. 1(3): p. 206-210.
232. Chen, in Chen, R., and Chen, C. (2016). “Exposure scenarios in the workplace and risk assessment of carbon nanomaterials,” in Biomedical Applications ad Toxicology of Carbon Nanomaterials, eds C. Chen and H. Wang (Weinheim: Wiley-VCH Verlag GmbH & Co. KGaA), 515-534. doi: 10.1002/9783527692866.ch16.
233. Nair, A. B. and S. Jacob, A simple practice guide for dose conversion between animals and human. Journal of basic and clinical pharmacy, 2016. 7(2): p. 27-31.
234. Montgomery, A. B., et al., A randomized double-blind placebo-controlled dose-escalation phase 1 study of aerosolized amikacin and fosfomycin delivered via the PARI investigational eFlow® inline nebulizer system in mechanically ventilated patients. Journal of aerosol medicine and pulmonary drug delivery, 2014. 27(6): p. 441-448.
235. Barth, P., et al., Single dose escalation studies with inhaled POL6014, a potent novel selective reversible inhibitor of human neutrophil elastase, in healthy volunteers and subjects with cystic fibrosis. Journal of Cystic Fibrosis, 2020. 19(2): p. 299-304.
236. Yamashita, C. M., et al., Matrix Metalloproteinase 3 Is a Mediator of Pulmonary Fibrosis. The American Journal of Pathology, 2011. 179(4): p. 1733-1745.
237. Matute-Bello, G., et al., An official American Thoracic Society workshop report: features and measurements of experimental acute lung injury in animals. American journal of respiratory cell and molecular biology, 2011. 44(5): p. 725-738.
238. Matute-Bello, G., C. W. Frevert, and T. R. Martin, Animal models of acute lung injury. American journal of physiology. Lung cellular and molecular physiology, 2008. 295(3): p. L379-L399.
239. Aeffner, F., B. Bolon, and I. C. Davis, Mouse models of acute respiratory distress syndrome: a review of analytical approaches, pathologic features, and common measurements. Toxicologic pathology, 2015. 43(8): p. 1074-1092.
240. Renne, R., et al., Recommendation of optimal method for formalin fixation of rodent lungs in routine toxicology studies. Toxicologic pathology, 2001. 29(5): p. 587-589.
241. Ingber, D., Integrins as mechanochemical transducers. Current Opinion in Cell Biology, 1991. 3(5): p. 841-848.
242. Wang, N., J. P. Butler, and D. E. Ingber, Mechanotransduction across the cell surface and through the cytoskeleton. Science, 1993. 260(5111): p. 1124-7.
243. Scuri, M., et al., Inhaled porcine pancreatic elastase causes bronchoconstriction via a bradykinin-mediated mechanism. Journal of Applied Physiology, 2000. 89(4): p. 1397-1402.
244. Mahmutovic Persson, I., et al., Longitudinal Imaging Using PET/CT with Collagen-I PET-Tracer and MRI for Assessment of Fibrotic and Inflammatory Lesions in a Rat Lung Injury Model. Journal of clinical medicine, 2020. 9(11): p. 3706.
245. Taylor, R. W., et al., Low-Dose Inhaled Nitric Oxide in Patients With Acute Lung Injury A Randomized Controlled Trial. JAMA, 2004. 291(13): p. 1603-1609.
246. Gelbard, M., et al., Clinical efficacy, safety and tolerability of collagenase clostridium histolyticum for the treatment of peyronie disease in 2 large double-blind, randomized, placebo controlled phase 3 studies. J Urol, 2013. 190(1): p. 199-207.
247. Saikumari, Y. K. and P. Balaram, An internally quenched fluorescent substrate for collagenase. Biopolymers, 2008. 90(2): p. 131-7.
248. Korkmaz, B., et al., Measurement of Neutrophil Elastase, Proteinase 3, and Cathepsin G Activities using Intramolecularly Quenched Fluorogenic Substrates, in Leucocytes: Methods and Protocols, R. B. Ashman, Editor. 2012, Humana Press: Totowa, N.J. p. 125-138.
249. Tokmina-Roszyk, M., et al., Development of a Förster resonance energy transfer assay for monitoring bacterial collagenase triple-helical peptidase activity. Anal Biochem, 2014. 453: p. 61-9.
250. Costas, B., et al., Efficacy and safety of collagenase clostridium histolyticum for Dupuytren disease nodules: a randomized controlled trial. BMC Musculoskeletal Disorders, 2017. 18(1): p. 374.
251. Lee, K. S., et al., Evaluation of bronchoalveolar lavage fluid from ARDS patients with regard to apoptosis. Respir Med, 2008. 102(3): p. 464-9.
252. Park, W. Y., et al., Cytokine balance in the lungs of patients with acute respiratory distress syndrome. Am J Respir Crit Care Med, 2001. 164(10 Pt 1): p. 1896-903.
253. Nakos, G., et al., Bronchoalveolar lavage fluid characteristics of early intermediate and late phases of ARDS. Alterations in leukocytes, proteins, PAF and surfactant components. Intensive Care Med, 1998. 24(4): p. 296-303.
254. Christner, P., et al., Collagenase in the lower respiratory tract of patients with adult respiratory distress syndrome. American Review of Respiratory Disease, 1985. 131(5): p. 690-695.

What is claimed is:

1. A method of treating acute lung injury in a patient, the method comprising:
administering by inhalation a therapeutically effective amount of one or more aerosolized collagenases to the lung tissue of the patient in need thereof such that the lung injury is treated;
wherein the one or more collagenases are administered at a dose of about 10-300 U/kg of body weight; and
wherein the one or more collagenases are administered to the lungs at the site of the proximate acute injury.

2. The method of claim 1, wherein the acute lung injury is acute respiratory distress syndrome (ARDS).

3. The method of claim 1, wherein the acute lung injury is selected from COPD, lung cancer, asthma, cystic fibrosis, emphysema, bronchitis, bronchiectasis, interstitial lung disease, interstitial fibrosis, bacterial pneumonia, viral pneumonia, fungal pneumonia, parasitic pneumonia, mycobacteria-caused pneumonia, occupational lung diseases, systemic lupus erythematosus, rheumatoid arthritis, scleroderma, dermatomyositis, mixed connective tissue disorder, vasculitis associated lung disease, sarcoid, or combinations thereof.

4. The method of claim 1, wherein the acute lung injury is the result of sepsis, pancreatitis, trauma to the lung tissue, pneumonia, aspiration, COVID-related illness, or combinations thereof.

5. The method of claim 1, wherein the administering is by nasal or oral inhalation.

6. The method of claim 1, wherein the collagenase acts as an enzymatic debrider, removing dead tissue from the lungs.

7. The method of claim 1, wherein the aerosolized collagenase has a diameter of about 0.1-10 μm.

8. The method of claim 1, wherein the dosage of aerosolized collagenase administered is about 0.1, 0.5, 1, 2, 3, 4, 5, 6, 7, 8, 9, or 10 mg/kg of patient body weight.

9. The method of claim 1, wherein the one or more collagenase is administered using an ultrasonic nebulizer.

10. The method of claim 1, wherein the patient is a human.

11. The method of claim 1, wherein the collagenase is selected from one or more of MMP1, MMP2, MMP3, MMP7, MMP8, MMP9, MMP10, MMP11, MMP12, MMP13, MMP14, MMP15, MMP16, MMP17, and microbial MMPs.

12. A method of preventing acute lung injury in a patient, the method comprising:
administering by inhalation a therapeutically effective amount of one or more aerosolized collagenases to the lung tissue of the patient such that the lung injury is prevented;
wherein the one or more collagenases are administered at a dose of about 10-300 U/kg of body weight; and
wherein the one or more collagenases are administered to the lungs at the site of the proximate acute injury.

13. The method of claim 12, wherein the patient is a human susceptible to developing acute lung injury.

14. The method of claim 12, wherein the acute lung injury is acute respiratory distress syndrome (ARDS).

15. The method of claim 12, wherein the acute lung injury is selected from COPD, lung cancer, asthma, cystic fibrosis, emphysema, bronchitis, bronchiectasis, interstitial lung disease, interstitial fibrosis, bacterial pneumonia, viral pneumonia, fungal pneumonia, parasitic pneumonia, mycobacteria-caused pneumonia, occupational lung diseases, systemic lupus erythematosus, rheumatoid arthritis, scleroderma, dermatomyositis, mixed connective tissue disorder, vasculitis associated lung disease, sarcoid, or combinations thereof.

16. The method of claim 12, wherein the administering is by nasal or oral inhalation.

17. The method of claim 12, wherein the collagenase is selected from one or more of MMP1, MMP2, MMP3, MMP7, MMP8, MMP9, MMP10, MMP11, MMP12, MMP13, MMP14, MMP15, MMP16, MMP17, and microbial MMPs.

18. The method of claim 12, wherein the aerosolized collagenase has a diameter of about 0.1-10 μm.

19. The method of claim 12, wherein the dosage of aerosolized collagenase administered is about 0.1, 0.5, 1, 2, 3, 4, 5, 6, 7, 8, 9, or 10 mg/kg of patient body weight.

20. A kit comprising:
a therapeutically effective amount of an aerosol form of collagenase; and
instructions for use.
